(19) United States
(12) Patent Application Publication
(10) Pub. No.: US 2008/0176813 A1
(43) Pub. Date: Jul. 24, 2008

(54) CARBOCYCLIC NUCLEOSIDES AND PROCESS FOR OBTAINING SUCH

(75) Inventors: Piet Herdewijn, Rotselaar (BE); Jing Wang, Sint-Niklaas (BE); Erik De Clercq, Lovenjoel (BE)

Correspondence Address: THE WEBB LAW FIRM, P.C., 700 KOPPERS BUILDING, 436 SEVENTH AVENUE, PITTSBURGH, PA, 15219, US

(73) Assignee: STICHTING REGA VZW, Leuven (BE)

(21) Appl. No.: 11/970,950

(22) Filed: Jan. 8, 2008

Related U.S. Application Data
(63) Continuation of application No. 10/070,791, filed on Aug. 5, 2002, now abandoned.
(60) Provisional application No. 60/153,086, filed on Sep. 10, 1999.
(60) Provisional application No. 60/153,087, filed on Sep. 10, 1999.
(60) Provisional application No. 60/214,897, filed on Jun. 29, 2000.

(51) Int. Cl. A61K 31/70; C07H 1/00; A61P 37/00

(52) U.S. Cl. 514/44; 536/22.1

(57) ABSTRACT

The present invention provides a six membered, at least partially unsaturated, carbocyclic nucleoside compound, including the (−) enantiomer, the (+) enantiomer, and pharmaceutically acceptable salts and esters thereof. The compounds are represented by formula (I), wherein Z represents one double bond in the six membered carbocylic ring, B is a heterocyclic ring, such as a pyrimidine or purine base, X is an azido, F or OR², R¹ and R² are the same or different and represent the same or different protecting groups, hydrogen, alkyl, alkenyl, acyl or phosphate moieties, and wherein the alkyl moiety is a saturated, optionally unsubstituted hydrocarbon having from 1 to 20 carbon atoms, the alkenyl moiety is an unsaturated congener of the alkyl group, and the acyl moiety is analkanoyl or aroyl moiety.

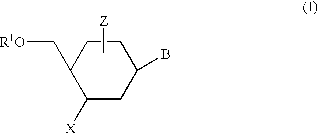

### CROSS-REFERENCE TO RELATED APPLICATIONS

This application is a continuation of co-pending application Ser. No. 10/070,791 filed Aug. 5, 2002, which is the national phase of PCT Application No. PCT/EP00/08882 filed 8 Sep. 2000 which, in turn, claimed priority to U.S. Provisional Application No. 60/153,086 filed Sep. 10, 1999; No. 60/153,087 filed Sep. 10, 1999; and No. 60/214,897 filed Jun. 29, 2000.

### BACKGROUND OF THE INVENTION

1. Field of the Invention

The invention relates to carbocyclic nucleoside analogues and their pharmaceutically acceptable salts, and to a method for the production of such, and to their use as anti-viral agents, amongst others.

2. Background of the Invention

Most antiviral compounds belong to the nucleoside field and the development of new modified nucleosides as antiviral agents is an active field of research. An object of the present invention is to provide an agent exhibiting pharmaceutical activity.

### SUMMARY OF THE INVENTION

According to a first aspect of the present invention there is provided a carbocyclic nucleoside analogue as described in claims 1-14.

These carbocyclic nucleotides exhibit good pharmaceutical activity.

According to a second aspect of the present invention there is provided a process for providing these carbocyclic compounds and intermediates thereof according to claims 15-24.

According to a further aspect of the present invention there is provided a carbocyclic compound according to any of the claims 24-35.

According to a further aspect of the present invention there is provided a pharmaceutical composition and the use of such, according to the claims 36-39.

Further aspects of the invention are detailed in claims 40-43.

The inventors have developed an enantioselective approach toward to the synthesis of (D)-cyclohexene nucleoside 4 (FIG. 3) using R-(−)-carvone (1) as inexpensive starting material. A sequence of chemical transformations led to the intermediate 2, possessing four chiral centers and, disregarding the protecting groups R₁-R₄, a plane of symmetry. Intermediate 2 allows for the synthesis of both the (D)- and the (L)-cyclohexene nucleosides 4 and 5, and 7 and 8, respectively.

The synthesis of (D)-adenine cyclohexene nucleoside 4 was accomplished by using a Mitsunobu type reaction on enol 3 to introduce the adenine base moiety on the cyclohexenyl ring. The corresponding guanine derivative 5 was synthesized in a similar way. Enol 3 was reacted with 2-amino-6-chloropurine in the presence of DEAD and triphenyl phosphine in dioxane to give 9 (FIG. 4), which was converted to 5 by treatment with TFA-H₂O 3:1. Under these reaction conditions the two TBDMS protecting groups were simultaneously removed. The overall yield starting from 3 was 46%. Analytically pure 5 was obtained by reversed-phase HPLC purification.

The synthesis of the (L)-cyclohexene nucleosides 7 and 8 was carried out by protection of the C4-CH₂OH and C5-OH groups (2b), followed by conversion of the OR₂ and OR₄ groups into enol 6. This compound was then used for the introduction of the base moiety according to the same strategy as used for the (D)-series. Intermediate 2b (R₁═R₃═H; R₂═R₄=TBDMS) was converted to dibenzoate 10 (FIG. 5) using standard reaction conditions (Bz₂O, DMAP, CH₂Cl₂, 98%). The equatorial C3-OTBDMS protecting group was selectively removed in the presence of the axial C1-OTBDMS to give 11 by using one equivalent of TBAF in THF at room temperature (74%). The selectivity of the desilylation reaction has been observed before^{13c}. The C3 alcohol 11 was converted to the corresponding mesylate 12, the C1-OTBDMS group was removed using TBAF to give the alcohol 13 (96%), which was oxidized using PDC in CH₂Cl₂. This oxidation was accompanied by simultaneous elimination of the C3-OMs group to afford directly the desired enone 15 (68%). Stereoselective reduction of enone 15 using NaBH₄ in the presence of CeCl₃.7H₂O in MeOH gave enol 6 (75%).

A Mitsunobu reaction was then applied for introduction of the base moiety (FIG. 6). Upon reaction of 6 with adenine in the presence of DEAD and PPh₃ in dioxane, the desired adenine derivative 16a was isolated in 40% yield, together with 15% of N7-isomer 16b (FIG. 6). Finally, removal of the benzoyl protecting groups using K₂CO₃ in MeOH gave the (L)-adenine cyclohexene nucleoside 7 in 72% yield. The corresponding (L)-guanine nucleoside 8 was synthesized in an analogous way. The enol 6 was treated with 2-amino-6-chloropurine under the same reaction condition as described above for 9, and the obtained 6-chloropurine 17 was converted to the guanine derivative 18 using TFA-H₂O 3:1 (58% yield from 6). Final deprotection was carried out by heating 18 in a saturated solution of NH₃ in MeOH in a sealed vessel for 2 days and reversed-phase HPLC purification gave pure (L)-guanine cyclohexene nucleoside 8 in 73% yield.

### Antiviral Activity

The anti-herpesvirus activity of D-cyclohexene G and L-cyclohexene G and the respective adenine analogues was determined in human embryonic skin muscle fibroblast (E₆SM: HSV-1, HSV-2) and in human embryonic lung (HEL) cells [varicella-zoster virus (VZV), cytomegalovirus (CMV)] (Table I). The source of the viruses and the methodology used to monitor antiviral activity have been previously described (De Clercq, E., Descamps, J., Verhelst, G., Walker, R. T., Jones, A. S., Torrence, P. F., Shugar, D. Comparative efficacy of antiherpes drugs against different strains of herpes simplex virus. J. Infect. Dis. 1980, 141, 563-574; De Clercq, E., Hóly, A., Rosenberg, I., Sakuma, T., Balzarini, J., Maudgal, P. C. A novel selective broad-spectrum anti-DNA virus agent. Nature, 1986, 323, 464-467). The antiviral activity was compared with the activity of known and approved antiviral drugs from which two with a purine base moiety (acyclovir, ganciclovir) and two with a pyrimidine base moiety (brivudine, cidofovir).

D-cyclohexene G as well as L-cyclohexene G did not show toxicity in four different cell lines (HeLa, Vero, E₆SM, HEL) (Table II), pointing to their selective antiviral mode of action, as reflected by the high selectivity index of the compounds (Table I). A salient feature is that the activity spectrum of both enantiomers is remarkably similar. Both compounds display the same activity against HSV-1 and HSV-2. Against VZV and CMV the potency of L-cyclohexene G is about 2-fold lower than that of D-cyclohexene G. Against HSV-1, the cyclohexene G nucleosides are as active as acyclovir and brivudin. Against HSV-2, their activity is very similar to that of acyclovir. The cyclohexene G nucleosides retain activity against the TK⁻ strains of HSV-1 and VZV, although the activity is reduced as compared to the activity against the wild type. The activity of D-cyclohexene G against TK⁺ and TK⁻ VZV strains is higher than the respective activities of acyclovir and brivudin against these viruses. D-cyclohexene G has the same activity against CMV as ganciclovir. In conclusion the activity spectrum of the cyclohexene nucleosides of the present invention is very similar to that of the known antiviral compounds possesing a guanine base moiety (acyclovir, ganciclovir). Both the D- and the L-enantiomers of cyclohexene G are antivirally active. The high selectivity indexes observed for D- and L-cyclohexene G indicates the utility of these compounds against herpesvirus infections.

D-cyclohexene G as well as L-cyclohexene G exhibited potent and selective anti-herpes virus (HSV1, HSV2, VZV, CMV) activity. Their activity spectrum is comparable to that of the known antiviral drugs acyclovir and ganciclovir. D- and L-cyclohexene G represent a very potent antiviral nucleosides containing a six-membered carbohydrate mimic. In contrast to the nucleosides with a cyclohexane, pyranose or hexitol ring, the cyclohexene nucleosides have a very flexible conformation. The inventors theorize that this flexibility may be an important structural determinant for their potent antiviral activity.

### BRIEF DESCRIPTION OF THE DRAWINGS

FIG. 1 is a reaction diagram showing the production of compounds C and D from starting materials A or B;

FIG. 2 is a formula showing the dependence of the conformation of α-alcohol 34 on solvent;

FIG. 3 is a reaction diagram showing an enantioselective synthesis of (D)-cyclohexene nucleoside 4 from R-(−)-carvone 1;

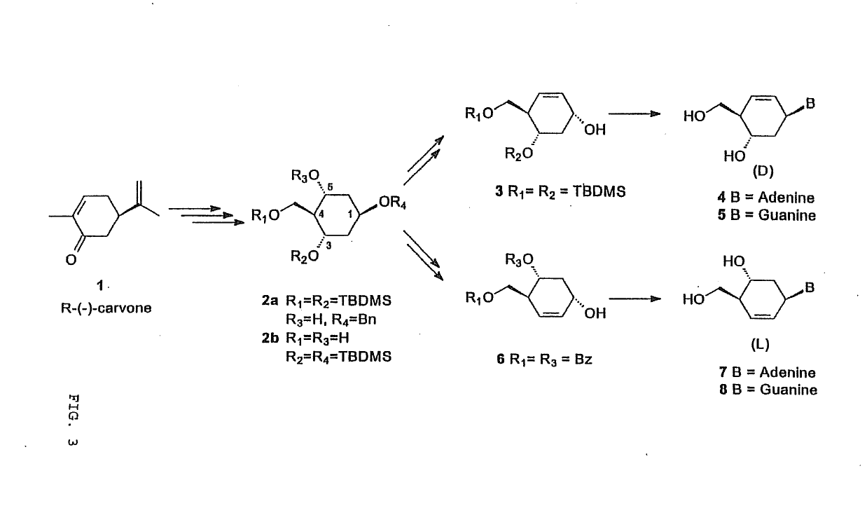

FIG. 4 is a reaction diagram of the production of compound 5 from compound 3;

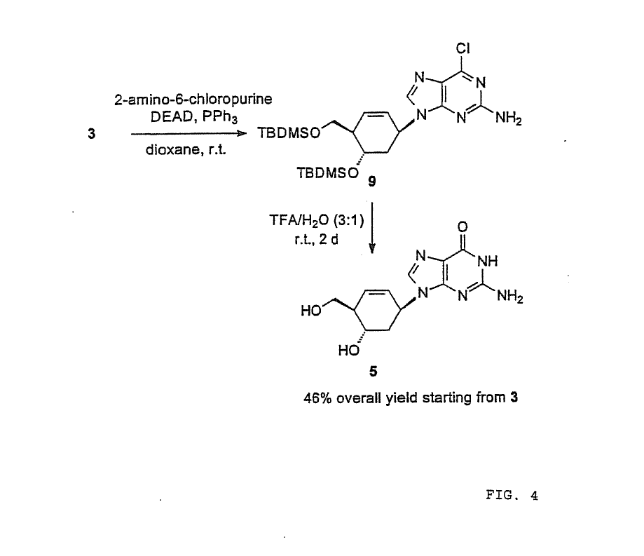

FIG. 5 is a reaction diagram of the conversion of intermediate 2b to dibenzonate 10 and further reaction to form compound 6;

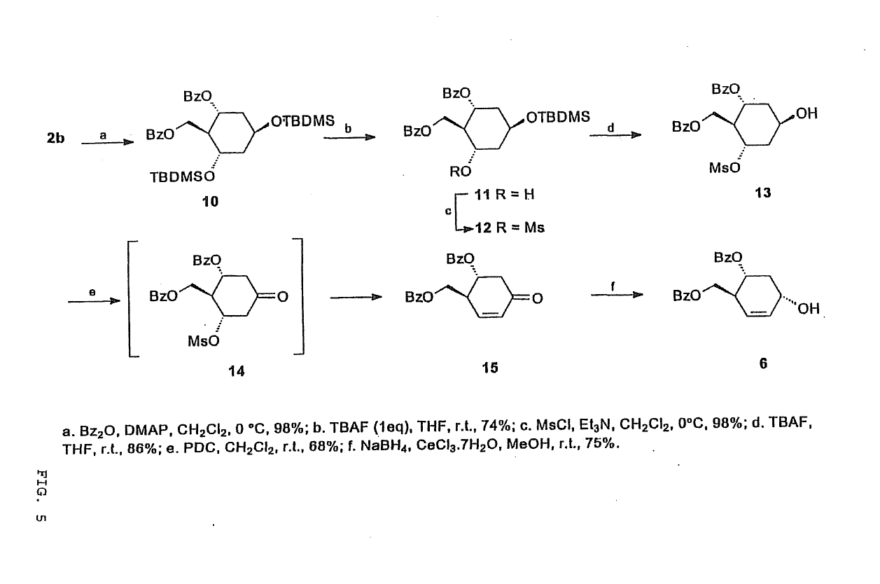

FIG. 6 is reaction diagram showing the use of a Mitsunobu reaction for the introduction of a base moiety, and the production of compounds 7 and 8 from compound 6;

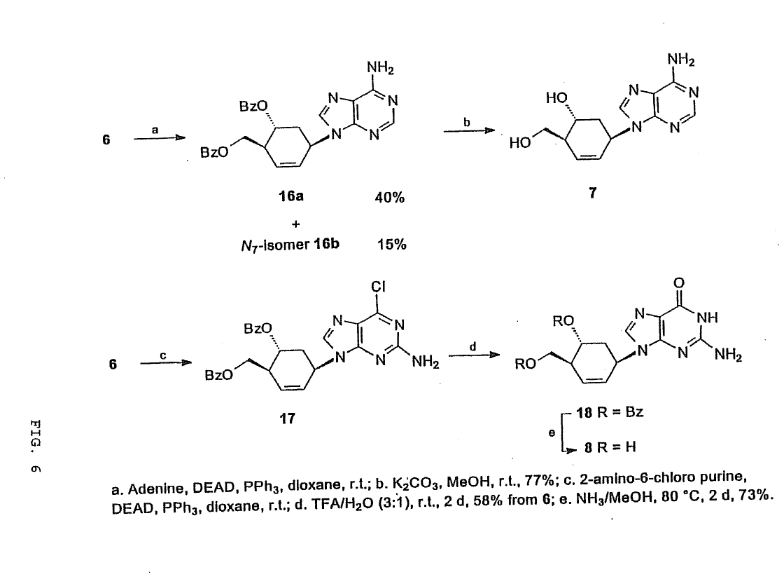

FIG. 7 is a reaction diagram showing the conversion of R-carvone to compounds 7a, 7b and 7c;

FIG. 8 is a reaction diagram showing the production of a mixture of cyclohexenes 11 and 13 from compound 7a;

FIG. 9 is a reaction diagram showing the production of compound 19 from compound 7b, and reaction strategies involving compound 19;

FIG. 10 is a reaction diagram showing the production of compound 34 from precursor 7c; and

FIG. 11 is a reaction diagram of the production of compounds D-2a and L-2a from intermediate 34.

### DETAILED DESCRIPTION OF THE INVENTION

### Experimental (1)

### General Methods

Melting points were determined in capillary tubes with a B [symbol] chi-Tottoli apparatus and are uncorrected. Ultraviolet spectra were recorded with a Philips PU 8740 UV/vis spectrophotometer. ¹H NMR and ¹³C NMR were determined with a 200 MHz Varian Gemini apparatus with tetramethylsilane as internal standard for the ¹H NMR spectra and DMSO-d₆ (39.6 ppm) or CDCl₃ (76.9 ppm) for the ¹³C NMR spectra (s=singlet, 3—doublet, dd—double doublet, t—triplet, br s—broad singlet, br d—broad doublet, m—multiplet). Liquid secondary ion mass spectra (LSIMS) with Cs⁺ as primary ion beam were recorded on a Kratos Concept IH (Kratos, Manchester, U.K.) mass spectrometer equipped with a MASPEC2 data system (Mass Spectrometry Services Ltd., Manchester, U.K.). Samples were directly dissolved in glycerol (gly)/thioglycerol(thgly)/m-nitrobenzyl alcohol (nba) and the secondary ions accelerated at 7 kV. Scans were performed at 10 s/decade from m/z 1000 down to m/z 50. Precoated Machery-Nagel Alugram SIL G/UV₂₅₄ plates were used for TLC (in solvent systems: A CH₂C1₂-MeOH 98:2, B CH₂C1₂-MeOH 9:1, C CH₂C1₂-EtOAc 4:1); the spots were examined with UV light and sulfuric acid/anisaldehyde spray. Elemental analyses were done at the University of Konstanz, Germany.

### 9-[(1S,4R,5S)-5-(tert-butyldimethylsilyloxy)-4-(tert-butyldimethylsilyloxymethyl)-2-cyclohexenyl]-2-amino-6-chloropurine (9)

To a mixture of 3 (130 mg, 0.35 mmol), 2-amino-6-chloropurine (119 mg, 0.70 mmol) and PPh₃ (184 mg, 0.70 mmol) in dry dioxane (7 mL) under N₂ at room temperature was added a solution of DEAD (110 μL, 0.70 mmol) in dry dioxane (3 mL) over a period of 1.5 hr. The reaction mixture was stirred at room temperature for two days and concentrated. The residue was chromatographed on silica gel (CH₂Cl₂-MeOH 50:1, then 20:1) to yield crude 9 (170 mg) as a yellow foam: ¹H NMR (CDCl₃) δ −0.10 (s, 3H), −0.04 (s, 3H), 0.07 (s, 6H), 0.82 (s, 9H), 0.90 (s, 9H), 2.04 (t, 2H, J=5.6 Hz), 2.27 (m, 1H), 3.66 (dd, 1H, J=9.9, 55.1 Hz), 3.77 (dd, 1H, J=9.9, 4.4 Hz), 3.98 (m, 1H), 5.21 (m, 1H), 5.43 (s, 2H, NH₂), 5.79 (dm, 1H, J=9.9 Hz), 6.00 (dm, 1H, J=9.9 Hz), 7.79 (s, 1H); ¹³C NMR (CDCl₃) δ −5.6 (q), −5.5 (q), −5.1 (q), −4.8 (q), 17.8 (s), 18.2 (s), 25.6 (q), 25.8 (q), 36.0 (t), 46.9 (d), 1049.2 (d), 62.9 (t), 64.6 (d), 124.4 (d), 125.4 (s), 134.4 (d), 141.3 (d), 151.1 (s), 153.3 (s), 159.1 (s);

### 9-[(1S,4R,5S)-5-hydroxy-4-hydroxymethyl-2-cyclohexenyl]guanine (5)

Crude 9 (170 mg) was treated with TFA-H₂O (3:1, 10 mL) at room temperature for two days. The reaction mixture was concentrated and co-evaporated with toluene (2×). The residue was chromatographed on silica gel (CH₂Cl₂-MeOH 10:1, then 1:1) to afford 5 (45 mg, 46% overall yield starting from 3): Mp >230° C.; UV λₘₐₓ (MeOH) 253 nm, ¹H NMR (CD₃OD) δ 1.94-2.27 (m, 3H), 3.77 (d, 2H, J=4.7 Hz), 3.85 (m, 1H), 5.17 (m, 1H), 5.88 (dm, 1H, J=10.2 Hz), 6.09 (dm, 1H, J=10.2 Hz), 7.73 (s, 1H); ¹³C NMR (CD₃OD) δ 37.1 (t), 47.7 (d), 50.6 (d), 63.1 (t), 64.8 (d), 125.8 (d), 135.4 (d), 138.5 (d); LISMS (THGLY/NBA) 278 (M+H)⁺; HRMS calcd for C₁₂H₁N₅O₃ (M+H)⁺ 278.1253, found 278.1270; Anal. Calcd for C₁₂H₁₅N₅O₃.0.77H₂O: C, 49.49; H, 5.73; N, 24.05. Found: C, 49.45; H, 5.55; N, 24.22.

### (1S,2S,3R,5R)-3-Benzoyl-2-benzoylmethyl-1,5-di(tert-butyldimethylsilyloxy)-cyclohexane (10)

To a solution of 2b (2.2 g, 5.64 mmol) in dry dichloromethane (20 mL) at 0° C. under N₂ was added DMAP (3.44 g, 28.2 mmol, 5 eq) and Bz₂O (3.83 g, 16.92 mmol, 3 eq) sequentially and in portions. After stirring at 0° C. for 2 hr, the reaction was quenched with ice. The reaction mixture was poured into CH₂Cl₂ (250 ml) and washed with water and brine, dried over Na₂SO₄ and concentrated. The crude product was chromatographed on silica gel (n-hexane-EtOAc 10:1) to yield 10 (3.3 g, 98%) as a light yellow oil.

¹H NMR (CDCl₃) δ 0.03 (s, 3H), 0.06 (s, 3H), 0.13 (s, 3H), 0.17 (s, 3H), 0.89 (s, 9H), 1.01 (s, 9H), 1.58 (m, 2H), 2.09 (m, 2H), 2.37 (m, 1H), 4.29 (br-s, 1H), 4.40 (td, 1H, J=10.3, 4.0 Hz), 4.49 (dd, 1H, J=11.4, 2.0 Hz), 104.61 (dd, 1H, J=11.4, 2.2 Hz), 5.66 (td, 1H, J=11.0, 4.6 Hz), 7.37-7.44 (m, 4H), 7.49-7.59 (m, 2H), 8.02 (m, 4H);

¹³C NMR (CDCl₃) δ −5.3 (q), −5.2 (q), −5.1 (q), −4.5 (q), 17.8 (s), 25.6 (q), 25.7 (q), 38.2 (t), 42.4 (t), 49.5 (d), 60.0 (t), 65.2 (d), 66.4 (d), 68.5 (d), 128.3 (d), 129.6 (d), 130.3 (s), 130.4 (s), 132.8 (d), 165.6 (s), 166.5 (s);

### (1S,2S,3R,5S)-3-Benzoyl-2-benzoylmethyl-5-(tert-butyldimethylsilyloxy)-cyclohexanol (11)

A solution of TBAF 1M in THF (5.38 ml, 5.38 mmol) was added slowly to a solution of 10 (3.23 g, 5.38 mmol) in THF (50 mL) at 0° C. The reaction mixture was stirred at 0° C. for 2 hr and further at room temperature for 3 hr. Ice was added and the reaction mixture was poured into EtOAc (300 ml) which was washed with NH₄Cl solution, water and brine, dried over Na₂SO₄ and concentrated. The crude product was purified on silica gel (n-hexane-EtOAc 5:1 then 1:1) to yield 11 (1.93 g, 74%) as a white foam.

¹H NMR (CDCl₃) δ 0.02 (s, 3H), 0.09 (s, 3H), 0.81 (s, 9H), 1.50-1.63 (m, 2H), 1.96 (m, 1H), 2.13 (m, 1H), 2.32 (m, 1H), 3.24 (d, 1H, J=4.8 Hz, —OH), 4.04 (m, 1H), 4.25 (m, 1H), 4.33 (dd, 1H, J=11.4, 2.2 Hz), 5.04 (dd, 1H, J=11.4, 2.2 Hz), 5.60 (td, 1H, J=11.0, 4.5 Hz), 7.36-7.60 (m, 6H), 8.06 (m, 4H);

¹³C NMR (CDCl₃) δ −5.2 (q), 17.6 (s), 25.4 (q), 38.2 (t), 40.8 (t), 50.4 (d), 59.8 (t), 64.1 (d), 66.1 (d), 68.4 (d), 128.4 (d), 129.6 (d), 129.8 (d), 130.4 (s), 133.0 (d), 133.2 (d), 165.6 (s), 167.6 (s);

LISMS (THGLY/TFA): 485 (M+H)⁺; HRMS calcd for C₂₇H₃₇O₆Si (M+H)⁺ 485.2359, found 485.2376.

### (1S,2S,3R,5S)-3-Benzoyl-2-benzoylmethyl-5-(tert-butyldimethylsilyloxy)-1-methanesulfonyloxy-cyclohexane (12)

To a solution of 11 (1.90 g, 3.92 mmol) in dry dichloromethane (20 mL) at 0° C. under N₂ was added slowly triethylamine (2.71 ml, 19.6 mmol, 5 eq) and MsCl (456 μl, 5.89 mmol, 1.5 eq) sequentially. After stirring at 0° C. for 1 hr, the reaction was quenched with ice. The reaction mixture was poured into CH₂Cl₂ (250 ml) and washed with a saturated NH₄Cl solution, water and brine, dried over Na₂SO₄ and concentrated. The residue was chromatographed on silica gel (n-hexane-EtOAc 1:1) to afford 12 (2.17 g, 98%) as a white foam.

¹H NMR (CDCl₃) δ 0.13 (s, 3H), 0.15 (s, 3H), 0.96 (s, 9H), 1.62 (td, 1H, J=12.0, 2.0 Hz), 1.85 (td, 1H, J=12.0, 2.0 Hz), 2.26-2.58 (m, 3H), 2.98 (s, 3H), 4.34 (m, 1H), 4.50 (dd, 1H, J=11.4, 2.0 Hz), 4.60 (dd, 1H, J=11.4, 2.0 Hz), 5.28 (td, 1H, J=11.1, 4.8 Hz), 5.69 (td, 1H, J=11.0, 4.8 Hz), 7.42 (m, 4H), 7.57 (m, 2H), 8.03 (m, 4H);

¹³C NMR (CDCl₃) δ −5.3 (q), −5.2 (q), 17.8 (s), 25.5 (q), 37.8 (t), 38.1 (q), 39.9 (t), 46.7 (d), 58.9 (t), 65.9 (d), 67.8 (d), 75.9 (d), 128.5 (d), 129.6 (d), 129.9 (s), 133.1 (d), 165.4 (s), 166.3 (s);

LISMS (THGLY/GLY): 563 (M+H)⁺; HRMS calcd for C₂₈H₃₉O₈SSi (M+H)⁺ 563.2135, found 563.2188.

### (1R,3S,4S,5R)-5-Benzoyl-4-benzoylmethyl-3-methanesulfonyloxy-cyclohexanol (13)

To a solution of 12 (2.15 g, 3.82 mmol) in THF (50 mL) at room temperature was added slowly a 1 M solution of TBAF (7.64 ml, 7.64 mmol, 2 eq) in THF. The reaction was stirred at room temperature for 2.5 hr and quenched with ice. After standard work-up and purification on silica gel (n-hexane-EtOAc 1:1), 13 (1.55 g, 86%) was obtained as a white foam.

¹H NMR (CDCl₃) δ 1.71 (td, 1H, J=12.1, 2.2 Hz), 1.90 (td, 1H, ⁻J=12.2, 2.3 Hz), 2.29-2.65 (m, 4H), 3.00 (s, 3H), 4.41 (m, 1H), 4.52 (dd, 1H, J=11.7, 2.8 Hz), 4.61 (dd, 1H, J=11.7, 2.8 Hz), 5.27 (td, 1H, J=10.6, 4.8 Hz), 5.65 (td, 1H, J=10.6, 4.7 Hz), 7.42 (m, 4H), 7.55 (m, 4H), 8.02 (m, 4H);

¹³C NMR (CDCl₃) δ 37.3 (t), 38.2 (q), 39.0 (t), 46.8 (d), 59.3 (t), 65.1 (d), 67.9 (d), 75.8 (d), 128.5 (d), 129.7 (d), 133.2 (d), 133.3 (d), 165.6 (s), 166.4 (s);

LISMS (THGLY/TFA): 449 (M+H)⁺; HRMS calcd for C₂₂H₂₅O₈S (M+H)⁺ 449.1270, found 449.1244.

### (4S,5R)-5-Benzoyl-4-benzoylmethyl-cyclohex-2-en-1-one (15)

A mixture of 13 (500 mg, 1.12 mmol) and PDC (2.1 g, 5.60 mmol, 5 eq) in dry CH₂Cl₂ (30 mL) was stirred vigorously at room temperature for 24 h. The reaction mixture was filtered through Celite® and washed with CH₂Cl₂. The filtrate was concentrated and the residue was chromatographed on silica gel (n-hexane-EtOAc 2:1, then 1:2) to yield starting material 13 (100 mg, 20%) and enone 15 (267 mg, 68%) as a light yellow oil.

¹H NMR (CDCl₃) δ 2.73 (dd, 1H, J=16.5, 8.8 Hz), 3.10 (dd, 1H, J=16.5, 4.4 Hz), 3.27 (m, 1H), 4.50 (dd, 1H, J=11.3, 4.7 Hz), 4.66 (dd, 1H, J=11.3, 5.5 Hz), 5.66 (ddd, 1H, J=8.8, 7.3, 4.4 Hz), 6.26 (dd, 1H, J=10.2, 2.2 Hz), 6.96 (dd, 1H, J=10.2, 3.3 Hz), 7.40-7.63 (m, 6H), 8.01 (m, 4H);

¹³C NMR (CDCl₃) δ 41.3 (d), 42.1 (t), 63.2 (t), 70.0 (d), 128.6 (d), 129.5 (2s), 129.7 (d), 129.8 (d), 131.4 (d), 133.5 (d), 146.5 (d), 165.5 (s), 166.4 (s), 195.8 (s);

### (1S,4S,5R)-5-Benzoyl-4-benzoylmethyl-cyclohex-2-en-1-ol (6)

To a solution of 15 (267 mg, 0.76 mmol) in MeOH (10 mL) at room temperature under N₂ was added CeCl₃.7H₂O (426 mg, 1.14 mmol, 1.5 eq). The mixture was stirred for 0.5 h and a clear solution was obtained. NaBH₄ (35 mg, 0.91 mmol, 1.2 eq) was added in portions and H₂ evolved. The reaction mixture was stirred for 1 h and quenched with ice. The mixture was stirred for 15 min and concentrated. The residue was distributed into EtOAc, washed with H₂O and brine, dried over Na₂SO₄ and concentrated. The residue was chromatographed on silica gel (n-hexane-EtOAc 10:1) to give 6 (200 mg, 75%) as a light yellow oil.

¹H NMR (CDCl₃) δ 1.77 (d, 1H, J=7.2 Hz), 1.93 (ddd, 1H, j-=12.1, 10.2, 8.0 Hz), 2.54 (ddd, 1H, J=12.1, 5.8, 3.3 Hz), 3.00 (m, 1H), 4.32 (dd, 1H, J=11.4, 5.5 Hz), 4.44 (dd, 1H, J=11.4, 5.5 Hz), 4.50 (m, 1H), 5.35 (ddd, 1H, J=10.2, 7.3, 2.9 Hz), 5.78 (dt, 1H, J=10.2, 1.8 Hz), 5.97 (dt, 1H, J=10.2, 2.5 Hz), 7.34-7.60 (m, 6H), 8.00 (m, 4H);

¹³C NMR (CDCl₃) δ 36.6 (t), 40.9 (d), 46.6 (t), 65.8 (d), 69.9 (d), 126.6 (d), 128.4 (d), 128.5 (d), 129.7 (d), 129.8 (s), 130.9 (s), 132.7 (d), 133.1 (d), 133.2 (d), 166.0 (s), 166.5 (s);

LISMS (THGLY/TFA): 353 (M+H)⁺; HRMS calcd for C₂₁H₂₁O₅ (M+H)⁺ 353.1389, found 353.1440.

### 9-[(1R,4S,5R)-5-Benzoyl-4-benzoylmethyl-2-cyclohexenyl]adenine (16a)

To a mixture of 6 (65 mg, 0.18 mmol), adenine (48 mg, 0.36 mmol, 2 eq) and PPh₃ (94 mg, 0.36 mmol, 2 eq) in dry dioxane (4 mL) under N₂ at room temperature was added a solution of DEAD (56 μL, 0.36 mmol, 2 eq) in dry dioxane (3 mL) over a period of 1 hr. The reaction mixture was stirred at room temperature overnight and concentrated. The residue was chromatographed on silica gel (CH₂Cl₂-MeOH 50:1, 20:1, 10:1) to yield 16a (33 mg, 40%) as a white solid.

UV λₘₐₓ (MeOH): 231 and 263 nm.

¹H NMR (CDCl₃) δ 2.48 (ddd, 1H, J=13.6, 8.3, 5.8 Hz), 52.57 (ddd, 1H, J=13.6, 6.0, 3.2 Hz), 4.50 (dd, 1H, J=10.4, 5.0 Hz), 4.63 (dd, 1H, J=10.4, 6.1 Hz), 5.53 (m, 2H), 5.92 (s, 2H), 6.09 (dm, 1H, J=10.0 Hz), 6.17 (dm, 1H, J=10.0 Hz), 7.41 (m, 4H), 7.57 (m, 2H), 7.86 (s, 1H), 8.04 (m, 4H), 8.35 (s, 1H);

¹³C NMR (CDCl₃) δ 32.4 (t), 40.6 (d), 48.7 (d), 64.3 (t), 68.2 (d), 120.1 (s), 126.8 (d), 128.5 (d), 128.6 (d), 129.6 (d), 129.7 (d), 131.0 (d), 133.4 (d), 138.8 (d), 149.8 (s), 153.1 (d), 155.8 (s), 165.8 (s), 166.5 (s);

LISMS (THGLY/NBA): 470 (M+H)⁺; HRMS calcd for C₂₆H₂₄N₅O₄ (M+H)⁺ 470.1828, found 470.1845.

### 9-[(1R,4S,5R)-5-hydroxy-4-hydroxymethyl-2-cyclohexenyl]adenine (7)

Compound 16a (33 mg, 0.07 mmol) was treated with anhydrous K₂CO₃ (100 mg) in MeOH (3 mL) at room temperature for 3 hr. Small portion of silica gel was added to the reaction mixture and the solvent was evaporated. The residue was chromatographed on silica gel (CH₂Cl₂-MeOH 10:1, 1:1) to give 7 (14 mg, 77%).

¹H NMR (CD₃OD) δ 2.02-2.32 (m, 3H), 3.79-3.90 (m, 3H), 5.35 (m, 1H), 5.93 (dm, 1H, J=9.9 Hz), 6.15 (dm, 1H, J=9.9 Hz), 8.09 (s, 1H), 8.21 (s, 1H);

¹³C NMR (CD₃OD) δ 37.2 (t), 47.7 (d), 51.1 (d), −63.0 (t), 64.7 (d), 120.6 (s), 125.4 (d), 136.0 (d), 141.6 (d), 30150.3 (s), 153.8 (d), 157.5 (s);

LISMS (THGLY/TFA): 262 (M+H)⁺; HRMS calcd for C₁₂H₁₆N₅O₂ (M+H)⁺ 262.1304, found 262.1323.

### 9-[(1R,4S,5R)-5-Benzoyl-4-benzoylmethyl-2-cyclohexenyl]guanine (18)

Compound 6 (160 mg, 0.45 mmol) was treated with 2-amino-6-chloropurine (153 mg, 0.90 mmol, 2 eq) in the presence of PPh₃ (235 mg, 0.90 mmol, 2 eq) and DEAD (140 μl, 0.90 mmol, 2 eq) in dry dioxane (12 mL) at room temperature overnight. After concentration and purification on silica gel (CH₂Cl₂-EtOAc 1:1), crude 17 (500 mg) was obtained, which was treated with CF₃COOH/H₂O (3:1, 12 mL) at room temperature for 2 days. The reaction mixture was concentrated and coevaporated with toluene. The residue was purified on silica gel (CH₂Cl₂-MeOH 20:1) to yield 18 (126 mg, 58% over two steps) as a white solid.

UV λₘₐₓ (MeOH): 251 and 256 nm.

¹H NMR (500 MHz, DMSO-d₆) δ 2.30 (ddd, 1H, J=13.6, 8.3, 5.9 Hz), 2.42 (ddd, 1H, J=13.6, 6.4, 3.2 Hz), 3.00 (m, 1H), 4.52 (m, 2H), 5.17 (m, 1H), 5.37 (m, 1H), 6.03 (dm, 1H, J=10.2 Hz), 6.11 (dm, 1H, J=10.2 Hz), 6.45 (s, 2H), 7.51 (m, 4H), 7.66 (m, 2H), 7.69 (s, 1H), 7.95 (m, 4H), 10.61 (s, 1H);

¹³C NMR (DMSO-d₆) δ 31.4 (t), 40.0 (d, overlapped with DMSO-d₆ peak), 47.9 (d), 64.4 (t), 68.5 (d), 116.9 (s), 127.0 (d), 128.9 (d), 129.3 (d), 129.4 (d), 130.2 (d), 133.6 (d), 135.7 (d), 150.9 (s), 153.8 (s), 156.9 (s), 165.3 (s), 165.8 (s);

LISMS (THGLY/GLY): 486 (M+H)⁺; HRMS calcd for C₂₆H₂₄N₅O₅ (M+H)⁺ 486.1777, found 486.1816;

UV (MeOH): 231, 256.

### 9-[(1R,4S,5R)-5-Hydroxy-4-hydroxymethyl-2-cyclohexenyl]guanine (8)

A mixture of 18 (85 mg) in an ammonium MeOH solution (75 mL) was sealed and heated at 80° C. for 2 days. After cooling to room temperature, the mixture was concentrated and the residue was purified by reverse HPLC (4% CH₃CN in water) to afford 8 (36 mg, 75%) as a white powder.

Mp: 255° C. (decomp.)

UV λₘₐₓ (MeOH)=254 nm.

¹H NMR (500 HMz, DMSO-d₆) δ 1.85 (m, 1H), 1.98 (m, 1H), 2.11 (m, 1H), 3.54 (dd, 1H, J=10.3, 5.5 Hz), 3.60 (dd, 1H, J=10.3, 4.8 Hz), 3.70 (m, 1H), 4.68 (br-s, 1H, —OH), 4.75 (br-s, 1H, —OH), 4.99 (m, 1H), 5.77 (dm, 1H, J=9.8 Hz), 5.97 (dm, 1H, J=9.8 Hz), 6.57 (s, 2H, —NH₂), 57.50 (s, 1H), 10.8 (br-s, 1H, —NH);

¹³C NMR (125 MHz, DMSO-d₆) δ 35.9 (t), 46.4 (d), 48.2 (d), 61.5 (t), 62.7 (d), 116.9 (s), 124.8 (d), 133.7 (d), 135.6 (d), 150.8 (s), 154.1 (s), 157.6 (s);

LISMS (THGLY/NBA) 278 (M+H)⁺; HRMS calcd for C₁₂H₁₆N₅O₃ (M+H)⁺ 278.1253, found 278.1247; Anal. Calcd for C₁₂H₁₅N₅O₃.1.5H₂O: C, 47.35; H, 5.96; N, 23.03. Found: C, 47.46; H, 5.64; N, 22.87.

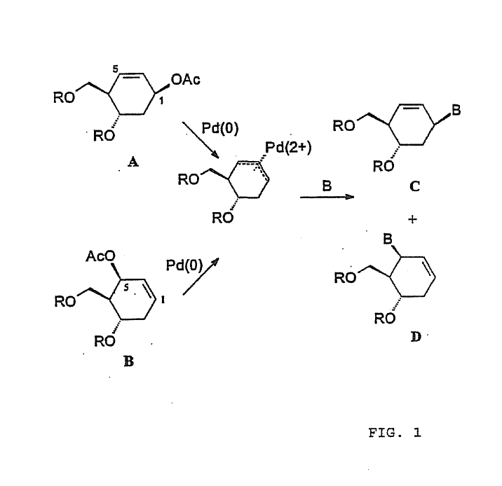

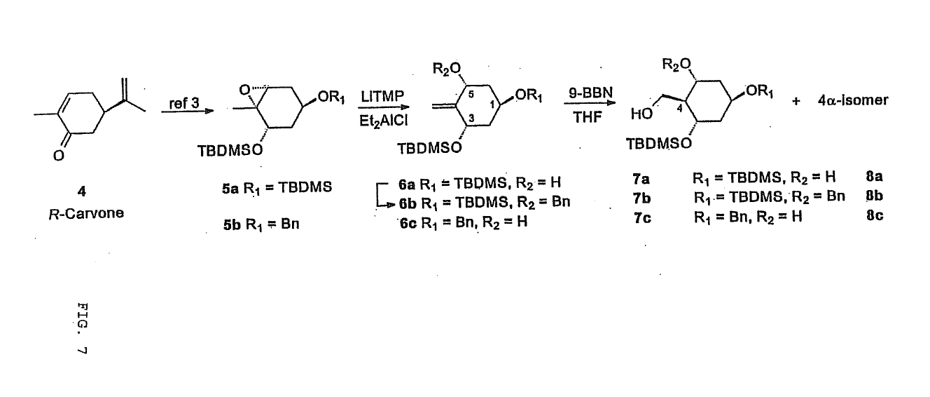

As detailed above, the inventors have developed an enantioselective approach to the synthesis of six-membered carbocyclic nucleosides of type 2b (R═OH) starting from (R)-(−)-carvone (4, FIG. 7, corresponding substantially with FIG. 1). A key step involving hydroboration of the exo double bond of cyclohexene 6b to afford hydroxymethyl substituted 7b with the correct stereochemistry at C4. Precursor 6a provided an ideal starting material for the synthesis of 3 as it had (1) a protected hydroxyl group at C3, (2) a protected hydroxyl substituent at C1, which at a final stage can be used to introduce a base moiety with retention of the configuration using Pd-chemistry, and (3) a free hydroxyl group at C5, which could be used to introduce the double bond.

The most straightforward approach seemed to introduce the C₅-C₆ double bond via conversion of the OH at C5 into a suitable leaving group, followed by a regioselective elimination. The latter might be achieved via a E₂-type elimination reaction by treatment with base, which requires a neighbouring hydrogen trans to the leaving group, only available on C6. In order to explore this strategy, alcohol 6a was converted into diol 7a via hydroboration using 9-BBN in THF. The reaction gave 7a as the major isomer, together with a small amount of epimer

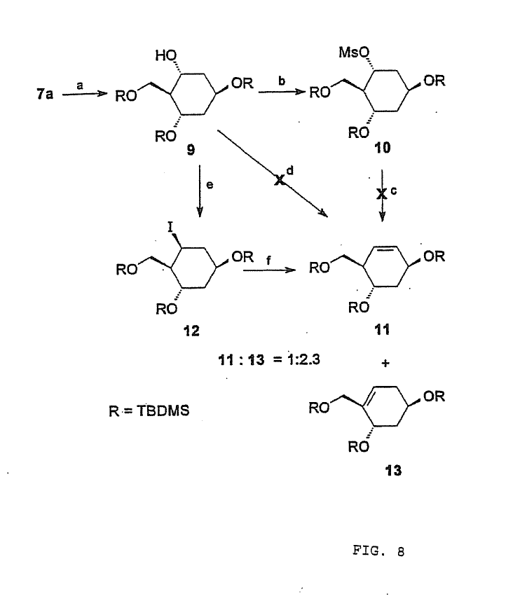

8a. The β-stereochemistry at C4 was easily established by NMR spectrometry. Selective protection of the primary hydroxyl group of 7a (TBDMSCl, imidazole, DMF) gave 9 (FIG. 8) and the leaving group was introduced (MsCl, Et₃N, dichloromethane) to give 10. However, upon treatment of mesylate 10 with DBU in toluene, cyclohexene 11 was not formed. More vigorous reaction conditions (KOH, H₂O-THF),⁵ likewise, failed to yield the unsaturated compound 11. Direct elimination of the 5-OH of 9 under Mitsunobu conditions (DEAD, PPh₃, THF)⁶ was also unsuccessful. 9 was converted into the β-iodide 12 (I₂, PPh₃, imidazole, toluene), with inversion of the stereochemistry at C5, followed by treatment with DBU in refluxing toluene. This reaction resulted in an inseparable mixture (yield 68%) of cyclohexenes 11 and 13 in a 1:2.3 ratio, respectively, in favour of the undesired regioisomer.

The inventors also investigated a different synthetic strategy, i.e. the construction of an allylic acetate of type A or B (FIG. 1) as intermediate for the Pd coupling reaction to introduce the base moiety.

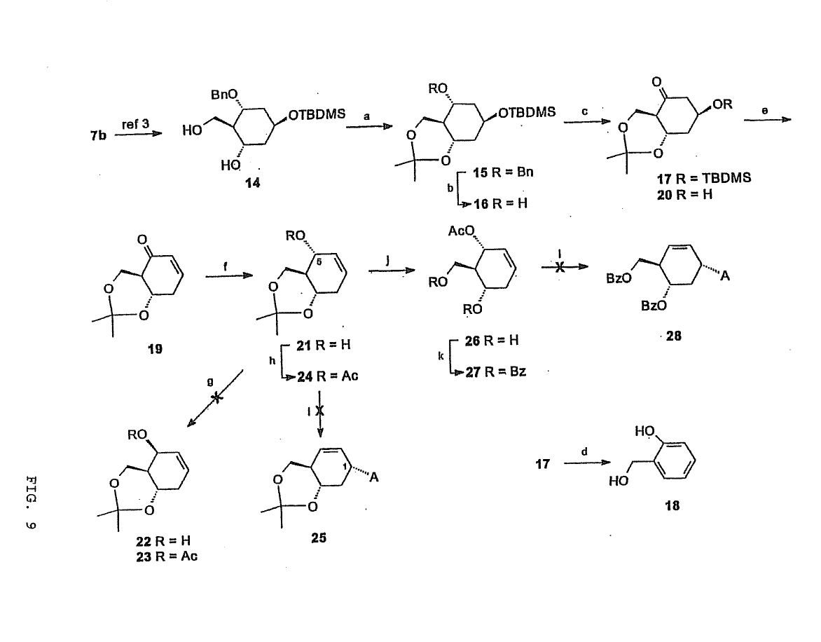

Diol 14 (FIG. 9) was protected as cyclic acetal 15 (2,2-dimethoxypropane, PPTS, acetone-THF), the Bn group was removed (10% Pd on carbon, HCOONH₄, MeOH, reflux) to give alcohol 16, and oxidation of the C5-OH (PDC, dichloromethane) provided ketone 17. Cleavage of the TBDMS ether using tetrabutylammonium fluoride (TBAF) in THF led mainly to diol 18. However, under neutral reaction conditions (KF, 18-crown-6, THF) the desired enone 19 was isolated in 62% yield; the p-hydroxy ketone intermediate 20 could not be detected. The critical reduction of enone 19 to the corresponding allylic alcohol 22 with β-oriented OH at C5 proved to be problematic, leading almost exclusively to the α-isomer 21 under the applied reaction conditions (NaBH₄, CeCl₃.7H₂O, MeOH and 9-BBN, THF). In an attempt to invert the stereochemistry at C5 of α-alcohol 21, the latter was subjected to a Mitsunobu type reaction (DEAD, PPh₃, AcOH), but the desired β-acetate 23 was not formed. However, compound 21 might be used to synthesize the x-analogue of the aforementioned cyclohexene nucleoside, interesting for as well conformational analysis as for determination of antiviral activity.

The intended Pd coupling reaction was investigated on the α-acetate 24, easily prepared from 21 (Ac₂O, DMAP, dichloromethane). When 24 was treated with the anion (NaH) of adenine in the presence of tetrakis(triphenylphosphine)palladium(0) in DMF-THF, only 24 was recovered and no trace of the 1α-adenine 25 could be detected. Reasoning that this failure might be due to the rigidity of the cyclic acetal present, 24 was treated with PPTS in MeOH to give diol 26, which was then converted into the corresponding dibenzoate 27 (Bz₂O, DMAP, dichloromethane). However, upon subjection of 27 to the same reaction conditions for coupling as applied above to 24, the expected 1α-adenine product 28 could not be isolated.

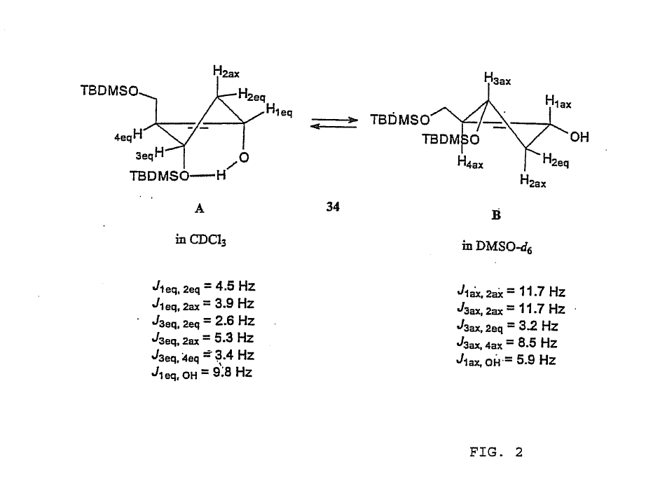

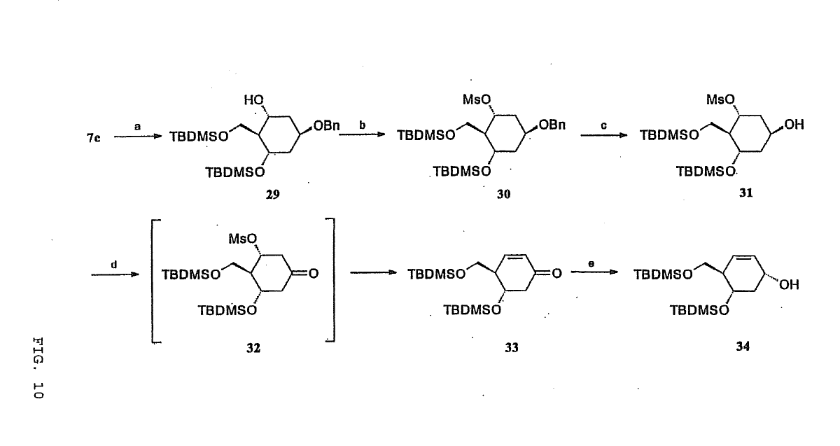

The above failure having exhausted the possibilities of the Pd coupling strategy, the most reliable alternative (for the introduction of the base moiety seemed) a Mitsunobu reaction was utilized, i.e. by substitution with inversion of the configuration of an α-oriented hydroxyl group at C1. Therefore the inventors had to synthesize an appropriately protected precursor 7c. Epoxide 5b (FIG. 7, R₁=Bn) was converted into 6c under the reported conditions (LiTMP and Et₂AlCl in benzene-toluene 1:1) in 79% yield. Hydroboration of 6c with 9-BBN in THF afforded 7c as major isomer (74%), together with its epimer 8c (20%). Similar to configurational assignment of 7a and 7b, the β-stereochemistry at C4 of 7c was established by ¹H-NMR. The primary hydroxyl group of 7c was selectively protected using 1.2 equivalents of TBDMSCl and imidazole in DMF t_e 2ve 29 (70%, FIG. 10), followed by conversion of the free alcohol at C5 into the corresponding mesylate 30 by treatment with MsCl and Et₃N in dichloromethane. Hydrogenolytic cleavage of the benzyl ether at C1 using 20% Pd(OH)₂ on carbon in the presence of cyclohexene in MeOH gave 31 in low yield (21%), which could be improved to 76% by the use of 10% Pd on carbon and HCOONH₄ in refluxing MeOH. Oxidation of alcohol 31 using PDC in dichloromethane gave a mixture of ketone 32 and enone 33 in a combined yield of 39%. However, using MnO₂ in dichloromethane, an incomplete but clean reaction took place. The ketone 32 was not isolated and enone 33 was obtained in 48% yield and recovered 31 (47%) could be recycled. Finally, enone 33 was reduced using NaBH₄ in the presence of CeCl₃.7H₂O in MeOH and gave the desired α-alcohol 34 as a single isomer in almost quantitative yield. The stereochemistry of 34 was confirmed by ¹H NMR spectral data. In CDCl₃ conformation A (FIG. 2), with the three substituents in a pseudoaxial position, predominates due to intramolecular hydrogen bonding between the C1-OH and C3-OTBDMS groups, while in DMSO-d₆ it adopts conformation B. This reflects the much lower axial-equatorial energy differences in cyclohexenes as compared to the corresponding cyclohexanes.

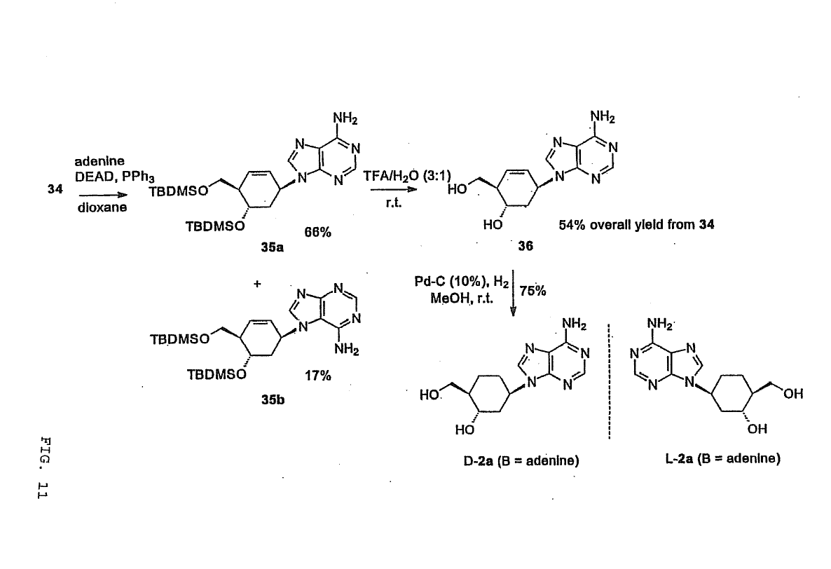

With intermediate 34 in hand, the base moiety (adenine) was introduced under Mitsunobu reaction conditions. Upon treatment of 34 with adenine in the presence of DEAD and PPh₃ in dioxane at room temperature for 1 day, 35a was isolated in 66% yield, together with 17% of its N₇-isomer 35b (FIG. 11). Complete deprotection of 35a using TBAF in THF at room temperature afforded the desired cyclohexene carbocyclic nucleoside 36 in almost quantitative yield. However, the compound was contaminated with tetrabutylammonium salts which could not be removed by standard chromatographic techniques. Recently Parlow et al. described a work-up procedure to remove tetrabutylammonium salts by the direct addition to the reaction mixture of mixed ion-exchange resins Amberlite® 15 and Amberlite® 15 in the Ca²⁺ form. Applied to the above TBAF reaction a complex mixture was obtained, giving 36 in low yield. In order to avoid the use of TBAF, Megron's method (Megron, G.; Vasquezy, F.; Galderon, G.; Cruz, R.; Gavino, R.; Islas, G. Synth. Commun. 1998, 26(16), 3021-3027) was used: compound 35a was treated with potassium tert-butoxide in DMF at room temperature. However, only a complex, reaction mixture was obtained, due to the strong basic character of the reaction conditions. Finally 35a was treated with a 3:1 mixture of TFA and H₂O at room temperature, which smoothly gave 36 in 54% overall yield starting from 34. According to our experience, this is the best procedure to cleave TBDMS ethers of this type of compound. Finally, 36 was purified by reversed-phase HPLC for analysis and determination of biological activity.

The above intermediate 36 (FIG. 11) gave the inventors the opportunity to obtain as yet 2a (B=adenine) in enantiopure form via reduction of the double bond. Thus, 36 was hydrogenated using H2 under atmospheric pressure in the presence 10% Pd on carbon in MeOH at room temperature to afford D-2a in 75% yield. The spectral data of D-2a were superimposable with those of a DL mixture of 2a. The enantiomeric purity of D-2a was examined by HPLC on a chiral column. The separation of a DL mixture of 2a together with the HPLC profile of D-2a synthesized by the above approach is depicted in reference 3c. Its enantiomeric purity proved 99%, at the same time establishing the high enantiomeric purity of 36.

The inventors have developed an enantioselective approach towards the synthesis of cyclohexene carbocyclic nucleosides starting from (R)-carvone 4. The synthetic methodology makes use of a Mitsunobu reaction as the key step to introduce the heterocyclic base moiety. The reaction proved to be efficient as well as chemo- and stereoselective, while the commonly applied palladium-mediated coupling strategy was unsuccessful. ¹H NMR and computation results show that in solution the synthesized adenine derivative 36 exists predominantly in a ³H₂ half-chair conformation with the adenine base orienting in a pseudoaxial position. The energy difference between ³H₂ and ²H₃ is, however, low. This compound may therefore be considered as a good mimic of a furanose nucleoside, showing two low energy conformations with a preference for the “3′-endo conformation”. This is also the preferred conformation of a hexitol nucleoside, in the ¹C₄ conformation. Moreover, the inventors theorize that the easy interconversion among both conformers might be a factor for antiviral activity.

### Experimental (2)

### (1R,3S, R)-5-Benzyloxy-3-(tert-butyldimethylsilyloxy)-2-methylenecyclohexanol (6c)

A solution of 2,2,6,6-tetramethylpiperidine (TMP, 27.3 mL, 162 mmol) in dry benzene (80 mL) and dry toluene (80 mL) was cooled to 0° C. under N₂ and a solution of n-BuLi in hexane (1.6 M, 64.8 mL, 162 mmol) was added dropwise. The resulting mixture was stirred at 0° C. for 10 min and a solution of Et₂AlCl (1.8 M, 90 mL, 162 mmol) in toluene was slowly added over a period of 1 hr. The reaction was stirred for an additional 30 min. A solution of 5b (14.1 g, 40.5 mmol) in benzene (30 mL) was added slowly. The reaction mixture was stirred at 0° C. for 3 h, then poured into an ice-cold NH₄Cl solution (300 mL). A 3 N HCl solution was added until a clear emulsion was obtained. The layers were separated and the aqueous layer was extracted with EtOAc (3×). The combined organic layers were washed with H₂O and brine, dried over Na₂SO₄ and concentrated. The residue was chromatographed on silica gel (n-hexane-EtOAc 10:1) to give 6c (10.2 g, 71%) as a light-yellow oil: ¹H NMR (CDCl₃) δ 0.09 (s, 6H), 0.92 (s, 9H), 1.90 (m, 4H), 2.69 (d, 1H, J=7.3 Hz, OH), 4.05 (m, 1H), 4.45 (m, 2H), 4.58 (s, 2H), 5.05 (s, 1H), 5.07 (s, 1H), 7.33 (m, 5H); ¹³C NMR (CDCl₃) δ −5.1 (q), 18.0 (s), 25.7 (q), 40.7 (t), 40.9 (t), 70.4 (d and t, overlapped), 70.8 (d), 71.3 (d), 107.1 (t), 127.5 (d), 128.4 (d), 138.7 (s), 150.7 (s).

### (1R,2s, 3S, 5R)-5-Benzyloxy-3- (tert-butyldimethylsilyloxy)-2-hydroxymethyl-cyclohexanol (7c) and its epimer 8c

To a solution of _{—}0 (10.8 g, 31.03 mmol) in dry THF (80 mL) at 0° C. under N₂ was added slowly a solution of 9-BBN in THF (0.5 M, 155 mL, 77.58 mmol). The reaction mixture was slowly warmed up to rt overnight. The reaction was cooled to 0° C. and treated sequentially with EtOH (30 mL), a 2 N NaOH solution (60 mL) and a 35% H₂O₂ solution (60 mL) under stirring. The resulting mixture was stirred at rt for 24 h, then poured into a mixture of EtOAc (300 mL) and H₂O (300 mL). The layers were separated and the aqueous layer was extracted with EtOAc (3×). The combined organic layers were washed with H₂O and brine, dried over Na₂SO₄ and concentrated. The crude product was separated on silica gel (n-hexane-EtOAc 5:1, then 1:1) to yield 7c (8.4 g, 74%) and epimer 8c (2.28 g, 20%) as a light-yellow oils.

7c: ¹H NMR (500 MHz, CDCl₃) δ 0.09 (2s, 6H), 0.91 (s, 9H), 1.52 (ddd, 1H, J=13.1, 10.1, 2.8 Hz), 1.54 (ddd, 1H, J=13.1, 10.1, 3.1 Hz), 1.69 (tdd, 1H, J=10.0, 7.5, 4.1 Hz), 2.10 (dt, 1H, J=13.1, 4.1 Hz), 2.16 (dt, 1H, J=13.1, 4.1 Hz), 2.71 (s, 1H), 3.11 (s, 1H), 3.78 (dd, 1H, J=10.1, 7.5 Hz), 3.85 (td, 1H, J=10.0, 4.2 Hz), 3.86 (m, 1H), 3.97 (br-td, 1H, J=10.1, 4.1 Hz), 4.04 (br-dd, 1H, J=10.1, 4.1 Hz), 4.51 (s, 2H), 7.26-7.37 (m, 5H); ¹³C NMR (CDCl₃) δ −5.0 (q), −4.3 (q), 17.8 (s), 25.7 (q), 38.1 (t), 38.4 (t), 53.2 (d), 63.4 (t), 68.0 (d), 69.4 (d), 70.3 (t), 72.4 (d), 127.4 (d), 127.6 (d), 128.4 (d), 138.7 (s); LISMS (THGLY): 367 (M+H)⁺; HPMS calcd for C₂₀H₃₅O₄Si (M+H)⁺ 367.2305, found 367.2341.

8c: ¹H NMR (CDCl₃) δ 0.07 (s, 3H), 0.08 (s, 3H), 0.85 (s, 9H), 1.40-1.87 (m, 3H), 2.25 (dm, 1H, J=13.2 Hz), 2.48 (dm, 1H, J=13.2 Hz), 3.69-4.20 (m, 6H), 4.33 (m, 1H), 4.53 (d, 1H, J=11.7 Hz), 4.62 (d, 1H, J=11.7 Hz), 7.33 (m, 5H), 8.79 (s, 1H); ¹³C NMR (CDCl₃) δ −5.6 (q), −5.0 (q), 21.9 (s), 25.5 (q), 39.2 (2t, overlapped), 45.9 (d), 61.3 (t), 69.0 (d), 69.4 (d), 70.4 (t), 70.8 (d), 127.6 (d), 127.7 (d), 128.4 (d), 138.6 (s); LISMS (THYLY): 367 (M+H)⁺; HRMS calcd for C₂₀H₃₅O₄Si (M+H)⁺ 367.2305, found 367.2335.

### (1R,2R,3S,5S)-5-Benzyloxy-3-(tert-butyldimethylsilyloxy)-2-(tert-butyldimethylsilyloxy methyl)cyclohexanol (29)

To a solution of 7c (2.5 g, 6.83 mmol) in DMF (50 mL) at rt were added imidazole (930 mg, 13.66 mmol) and TBDMSCl (1.23 g, 8.2 mmol) in portions. The reaction was stirred at rt overnight and quenched with ice. The resulting mixture was evaporated to remove DMF and the residue was partitioned between EtOAc and H₂O. The layers were separated and the aqueous layer was extracted with EtOAc (2×). The combined organic layers were washed with H₂O and brine, dried over Na₂SO₄ and concentrated. The residue was chromatographed on silica gel (n-hexane-EtOAc 5:1) to yield 29 (2.28 g, 70%) as a light-yellow oil: ¹H NMR (CDCl₃) δ 0.05, 0.06, 0.09 (3s, 12H), 0.89, 0.91 (2s, 18H), 1.53 (m, 2H), 1.72 (qd, 1H, J=9.5, 4.4 Hz), 2.11 (m, 2H), 3.67 (t, 1H, J=9.5 Hz), 3.78 (td, 1H, J=9.5, 4.4 Hz), 3.87 (m, 1H), 4.01 (m, 1H), 4.16 (dd, 1H, J=9.5, 4.4 Hz), 4.46 (d, 1H, J=15.2 Hz), 4.48 (d, 1H, J=15.2 Hz), 7.33 (m, 5H); ¹³C NMR (CDCl₃) δ −5.7 (q), −5.1 (q), −4.3 (q), 17.8, 18.0 (2s), 25.7 (2q), 37.0 (t), 38.4 (t), 52.2 (d), 66.2 (t), 67.2 (d), 70.1 (t and d overlapped), 72.4 (d), 127.3 (d), 127.4 (d), 128.4 (d), 138.9 (s); LISMS (GLY): 481 (M+H)⁺; HRMS cald for C₂₆H₄₉O₄Si: (M+H)⁺ 481.3169, found 481.3199.

### (1R,2R,3S,5S)-5-Benzyloxy-2-(tert-butyldimethylsilyloxymethyl)-3-(tert-butyldimethylsilyoxy)-1-methanesulfonyloxy-cyclohexane (30)

To a solution of 29 (5.4 g, 11.25 mmol) in CH₂Cl₂ (120 mL) at 0° C. was added triethylamine (7.8 mL, 56.25 mmol), followed by dropwise addition of MsCl (1.3 mL, 16.87 mmol). The reaction was stirred at 0° C. for 1 h and treated with ice. The resulting mixture was separated and the aqueous layer was extracted with CH₂Cl₂ (2×). The combined organic layers were washed with a diluted HCl solution, H₂O and brine, dried over Na₂SO₄ and concentrated. The residue was chromatographed on silica gel (n-hexane-EtOAc 5:1) to afford 30 (5.81 g, 92%) as a white solid: mp 100-101° C.; ¹H NMR (CDCl₃) δ 0.08 (2s, 12H), 0.89 (s, 9H), 0.90 (s, 9H), 1.43 (ddd, 1H, J=13.9, 10.0, 2.8 Hz), 1.62 (tt, 1H, J=10.2, 2.0 Hz), 1.71 (ddd, 1H, J=12.8, 10.6, 2.2 Hz), 2.24 (br-d, 1H, J=13.9 Hz), 2.69 (br-d, 1H, J=12.8 Hz), 3.01 (s, 3H), 3.74 (dd, 1H, J=9.9, 2.2 Hz), 3.89 (m, 1H), 3.91 (dd, 1H, J=9.9, 1.8 Hz), 4.19 (td, 1H, J=10.0, 4.7 Hz), 4.45 (d, 1H, J=12.0 Hz), 4.57 (d, 1H, J=12.0 Hz), 5.13 (td, 1H, J=10.6, 4.8 Hz), 7.33 (m, 5H); ¹³C NMR (CDCl₃) δ −5.6 (q), −5.3 (q), −4.6 (q), −3.7 (q), 17.9 (s), 25.8 (q), 35.5 (t), 38.5 (t), 38.8 (q), 51.8 (d), 56.9 (t), 65.1 (d), 70.1 (t), 72.0 (d), 77.5 (d), 127.4 (d), 128.4 (d), 138.5 (s); LISMS (GLY/NBA) 559 (M+H)⁺; HRMS calcd for C₂₇H₅₁O₆SSi₂ (M+H)⁺ 559.2945, found 559.2979; Anal. Calcd for C₂₇H₅₁O₆SSi₂: C, 58.02; H, 9.02. Found: C, 57.96; H, 8.82.

### (1S,3R,4R,5S)-4-tert-Butyldimethylsilyloxymethyl-5-tert-butyldimethylsilyloxy-3-methanesulfonyloxy-cyclohexanol (31)

A mixture of 30 (3.5 g, 6.27 mmol), Pd/C (10%, 4.4 g) and HCOONH₄ (2.2 g) in MeOH (100 mL) was refluxed and 2×1.1 g of HCOONH₄ were added every 3 h interval. The reaction was refluxed until all the starting material was consumed (total 14 h). After cooling to rt, the reaction mixture was filtered through Celite® and the residue was washed with CH₂Cl₂ (3×). The filtrate was concentrated to afford crude 31 (2.83 g, 97%) as a white solid, which was used as such for the next step: mp 135-137° C.; ¹H NMR (CDCl₃) δ 0.08, 0.09 (2s, 12H), 0.89 (s, 9H), 0.92 (s, 9H), 1.43-1.68 (m, 3H), 1.83 (ddd, 1H, J=13.2, 10.6, 2.8 Hz), 2.07 (br-d, 1H, J=13.2 Hz), 2.44 (br-d, 1H, J=13.2 Hz), 3.02 (s, 3H), 3.72 (dd, 1H, J=10.0, 2.4 Hz), 3.90 (dd, 1H, J=10.0, 2.4 Hz), 4.19 (td, 1H, J=10.6, 4.1 Hz), 4.26 (m, 1H), 5.14 (td, 1H, J=10.6, 4.7 Hz); ¹³C NMR (CDCl₃) δ −5.6 (q), −5.3 (q), −4.7 (q), −3.8 (q), 17.9 (s), 25.8 (q), 38.8 (q), 38.9 (t), 40.8 (t), 51.7 (d), 57.1 (t), 64.9 (d), 65.5 (d), 77.3 (d); LISMS (GLY/NBA) 469 (M+H)⁺; HRMS calcd for C₂₀H₄₅O₆SSi₂ (M+H)⁺ 469.2475, found 469.2453; Anal. Calcd for C₂₀H₄₅O₆SSi₂: C, 51.24; H, 9.46. Found: C, 51.24; H, 9.36.

### (4R,5S)-4-tert-Butyldimethylsilyloxymethyl-5-tert-butyldimethylsilyloxy-cyclohex-2-en-1-one (33)

A mixture of crude 31 (2.83 g, 6.27 mmol) and MnO₂ (13.6 g, 156.8 mmol) in dry CH₂Cl₂ (100 mL) was stirred vigorously at rt for 21 h. The reaction mixture was filtered through Celite® and washed with CH₂Cl₂. The filtrate was concentrated and the residue was chromatographed on silica gel (n-hexane-EtOAc 5:1, then 1:2) to yield starting material 30 (1.56 g, 53%) and enone 33 (920 mg, 40% over two steps) as a light-yellow oil (solid upon storing in the refrigerator): ¹H NMR (CDCl₃) δ 0.07 (s, 12H), 0.89 (s, 18H), 2.50 (m, 1H), 2.46 (dd, 1H, J=16.1, 10.6 Hz), 2.72 (dd, 1H, J=16.1, 4.8 Hz), 3.73 (dd, 1H, J=9.9, 5.6 Hz), 3.85 (dd, 1H, J=9.9, 4.4 Hz), 4.09 (ddd, 1H, J=10.6, 8.1, 4.8 Hz), 6.06 (dd, 1H, J=10.2, 2.6 Hz), 6.88 (dd, 1H, J=10.2, 2.6 Hz); ¹³C NMR (CDCl₃) δ −5.6 (q), −5.5 (q), −5.1 (q), −4.4 (q), 17.8 (s), 18.2 (s), 25.6 (q), 25.8 (q), 47.1 (t), 48.0 (d), 61.8 (t), 68.0 (d), 130.2 (d), 150.6 (d), 199.0 (s); LISMS (THGLY/NBA) 371 (M+H)⁺; HRMS calcd for C₁₉H₃₉O₃Si₂ (M+H)⁺ 371.2438, found 371.2432.

### (1R,4R,5S)-5-(tert-Butyldimethylsilyloxy)-4-(tert-butyldimethylsilyloxymethyl)-cyclohex-2-en-1-ol (34)

To a solution of 33 (920 mg, 2.49 mmol) in MeOH (35 mL) at rt under N₂ was added CeCl₃.7H₂O (1.39 g, 3.73 mmol). The mixture was stirred for 0.5 h and a clear solution was obtained. NaBH₄ (113 mg, 2.99 mmol) was added in portions and H₂ evolved. The reaction mixture was stirred for 1 h and quenched with H₂O. The mixture was stirred for 15 min and concentrated. The residue was diluted with EtOAc, washed with H₂O and brine, dried over Na₂SO₄ and concentrated. The residue was chromatographed on silica gel (n-hexane-EtOAc 10:1) to give 34 (844 mg, 91%) as a colourless oil: ¹H NMR (500 MHz, CDCl₃) δ 0.04 (s, 3H), 0.05 (s, 3H), 0.10 (s, 3H), 0.11 (s, 3H), 0.89 (s, 9H), 0.90 (s, 9H), 1.94 (ddd, 1H, J=13.7, 5.3, 3.9 Hz), 1.99 (ddd, 1H, J=13.7, 4.5, 2.6 Hz), 2.36 (m, 1H), 2.94 (d, 1H, J=9.8 Hz), 3.38 (dd, 1H, J=10.1, 7.8 Hz), 3.56 (dd, 1H, J=10.1, 5.0 Hz), 4.09 (pseudo sext, 1H, J=9.8, 4.5, 4.0, 3.9 Hz), 4.20 (pseudo pent, 1H, J=5.3, 3.4, 2.6 Hz), 5.61 (dd, 1H, J=10.0, 3.9 Hz), 5.95 (ddd, 1H, J=10.0, 4.0, 1.8 Hz); ¹³C NMR (CDCl₃) δ −5.5 (q), −5.4 (q), −4.9 (q), −4.8 (q), 18.0 (s), 18.3 (s), 25.8 (q), 25.9 (q), 35.6 (t), 46.5 (d), 63.5 (t), 64.8 (d), 67.7 (d), 127.0 (d), 131.1 (d); LISMS (THGLY/NBA) 373 (M+H)⁺; HRMS calcd for C₁₉H₄₀O₃Si₂ (M+H)⁺ 373.2594, found 373.2626; Anal. Calcd for C₁₉H₄₀O₃Si₂: C, 61.23; H, 10.82. Found: C, 61.34; H, 10.83.

### 9-[(1S,4R,5S)-5-(tert-Butyldimethylsilyloxy)-4-(tert-butyldimethylsilyloxymethyl)-2-cyclohexenyl]adenine (35a)

To a mixture of 34 (660 mg, 1.774 mmol), adenine (480 mg, 3.55 mmol) and PPh₃ (931 mg, 3.55 mmol) in dry dioxane (20 mL) under N₂ at rt was added a solution of DEAD (565 μL, 3.55 mmol) in dry dioxane (10 mL) over a period of 45 min. The reaction mixture was stirred at rt overnight, concentrated and the residue was chromatographed on silica gel (CH₂Cl₂-MeOH 50:1, then 20:1) to yield crude 35a (960 mg) as a yellow foam: ¹H NMR (CDCl₃) δ −0.12 (s, 3H), −0.06 (s, 3H), 0.10 (s, 3H), 0.11 (s, 3H), 0.83 (s, 9H), 0.94 (s, 9H), 2.01-2.25 (m, 2H), 2.32 (m, 1H), 3.73 (dd, 1H, J=9.9, 4.8 Hz), 3.82 (dd, 1H, J=9.9, 4.4 Hz), 3.97 (ddd, 1H, J=10.2, 7.0, 4.0 Hz), 5.37 (m, 1H), 5.73 (s, 2H), 5.88 (ddd, 1H, J=9.9, 3.7, 2.5 Hz), 6.06 (ddd, 1H, J=9.9, 2.2, 1.1 Hz), 7.86 (s, 1H), 8.39 (s, 1H); ¹³C NMR (CDCl₃) δ −5.5 (q), −5.4 (q), −5.0 (q), −4.6 (q), 17.8 (s), 18.3 (s), 25.6 (q), 25.9 (q), 36.5 (t), 47.2 (d), 49.6 (d), 62.9 (t), 64.5 (d), 120.2 (s), 124.4 (d), 134.9 (d), 139.9 (d), 149.8 (s), 153.0 (d), 155.5 (s); LISMS (THGLY/NBA) 490 (M+H)⁺; HRMS calcd for C₂₄H₄₄N₅O₂Si₂ (M+H)⁺ 490.3034, found 490.3058.

### 9-[(1S,4R,5S)-5-Hydroxy-4-hydroxymethyl-2-cyclohexenyl]adenine (36)

Crude 35a was treated with TFA-H₂O (3:1, 40 mL) at rt overnight. The reaction mixture was concentrated and co-evaporated with toluene (2×). The residue was chromatographed on silica gel (CH₂Cl₂-MeOH 20:1, then 5:1) to afford 36 (149 mg, 54% over two steps):

Mp 90-92° C.; ¹H NMR (CD₃OD) δ 2.01-2.33 (m, 3H), 3.80 (d, 2H, J=4.8 Hz), 3.84 (m, 1H), 5.33 (m, 1H), 5.94 (ddd, 1H, J=9.9, 3.7, 2.6 Hz), 6.13 (ddd., 1H, J=9.9, 2.5, 1.4 Hz), 8.09 (s, 1H), 8.21 (s, 1H); ¹³C NMR (CD₃OD) δ 37.3 (t), 47.9 (d), 51.1 (d), 63.1 (t), 64.7 (d), 120.6 (s), 125.3 (d), 136.1 (d), 141.6 (d), 150.4 (s), 153.7 (d), 157.5 (s); UV λₘₐₓ (MeOH)=260 nm; LISMS (THGLY/NBA) 262 (M+H)⁺; HRMS calcd for C₁₂H₁₆N₅O₂ (M+H)⁺ 262.1304, found 262.1359; Anal. Calcd for C₁₂H₁₆N₅O₂.0.7H₂O: C, 52.62; H, 6.04; N, 25.57. Found: C, 52.62; H, 5.95; N, 25.77.

### 9-[(1R,3S,4R)-3-Hydroxy-4-hydroxymethylcyclohexanyl]adenine (2a)

A mixture of 36 (45 mg, 0.17 mmol) and Pd/C (10%, 40 mg) in MeOH (5 mL) was stirred under H₂ at rt for 24 h. The reaction mixture was cooled to rt and filtered through Celite® and washed with MeOH. The filtrate was concentrated and theireÿidue was purified by reversed-phase HPLC (5% CH₃CN in H₂O) to yield 2a (35 mg, 78%) as a white foam: ¹H NMR (CD₃OD) δ 1.71 (m, 1H), 1.87-2.18 (m, 5H), 2.39 (m, 1H), 3.69 (dd, 1H, J=14.0, 7.3 Hz), 3.74 (dd, 1H, J=14.0, 6.9 Hz), 4.12 (m, 1H), 4.87 (m, 1H, overlapped with HOD), 8.18 (s, 1H), 8.21 (s, 1H); ¹³C NMR (CD₃OD) δ 22.6 (t), 28.7 (t), 36.1 (t), 53.6 (d), 51.9 (d), 63.3 (t), 68.4 (d), 120.4 (s), 141.1 (d), 150.6 (s), 153.5 (d), 157.4 (s); LISMS (THGLY/NBA) 264 (M+H)⁺; HRMS calcd for C₁₂H₁₈N₅O₂ (M+H)⁺ 264.1460, found 264.1449.

FIG. 1. Mechanism of Pd (0) coupling reaction which may yield the desired compound C.

FIG. 2. ¹H NMR experiment demonstrates the solvent-dependent conformational equilibrium of compound 34.

FIG. 8 (a) TBDMSCl (1.2 eq), imidazole (2 eq), DMF, r.t., 48% starting from 6a; (b) MsCl, Et₃N, CH₂Cl₂, 0° C., 93%; (c) DBU, toluene, or KOH, H₂O/THF; (d) DEAD, PPh₃, THF; (e) I₂, PPh₃, imidazole, toluene, reflux, 34%; (f) DBU, toluene, reflux, 68%.

FIG. 9 (a) (CH₃)₂C(OCH₃)₂, PPTS, acetone/THF (1:2), r.t., 94%; (b) Pd—C (10%), HCOONH₄, MeOH, reflux, 100%; (c) PDC, CH₂Cl₂, r.t., 94%; (d) TBAF, THF, r.t.; (e) KF, 18-Crown-6, THF, r.t., 62% 19; (f) CeCl₃.7H₂O, NaBH₄, MeOH, 90%; (g) PPh₃, DEAD, AcOH, THF; (h) Ac₂O, DMAP, CH₂Cl₂, 0° C., 95%; (i) Adenine, NaH, Pd(PPh₃)₄, DMF/THF; (j) PPTS, MeOH, r.t., 59%; (k) Bz₂O, DMAP, CH₂Cl₂, 0° C., 95%.

FIG. 10 (a) TBDMSCl (1.2 eq), imidazole (1.5 eq), DMF, r.t., 70%; (b) MsCl, Et₃N, CH₂Cl₂, 0° C., 100%; (c) Pd—C (10%), HCOONH₄, MeOH, reflux, 76%; (d) MnO₂, CH₂Cl₂, r.t., 48% and 47% recovery of 31; (e) NaBH₄, CeCl₃.7H₂O, MeOH, 0° C.→+r.t., 100%.

### Sodium salt of Ethyl-β-hydroxyacrylate sodium salt (1)

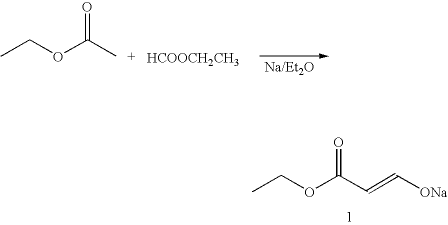

In a 1 L three necked flask, under inert atmosphere and equipped with an addition funnel, a well stirred suspension of fresh-sodium pieces (23.0 g, 1.0 mol) in dry diethyl ether (400 mL) was prepared. A mixture of ethyl acetate (88.0 g, 1.0 mol) and ethyl formate (74.0 g, 1.0 mol) was added dropwise over a period of 45 minutes. Stirring was continued for an additional 14 hours using an ice bath, avoiding the reaction to become too vigorous. The resulting suspension was kept in the refrigerator for 8 hours, after which it was filtered, washed with dry diethyl ether (100 mL) and dried in vacuo to obtain 1 as a pale yellow solid (85.0 g, 61% yield).

### Ethyl β-acetoxyacrylate [cis (2′)+trans (2)]

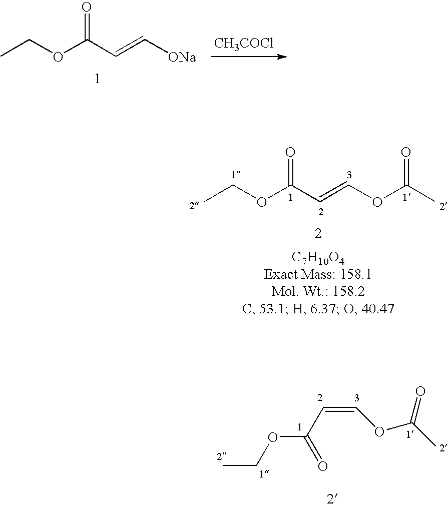

In a 2 L flask on an ice-bath, under an inert atmosphere, a well stirred suspension of the sodium salt 1 (85.0 g, 616 mmol) was prepared in dry diethyl ether (850 mL), to which acetyl chloride (52.9 mL, 58.2 g, 739 mmol) was added dropwise over 15 minutes. The mixture was stirred for an additional 6 hours, after which it was neutralized with a saturated aqueous solution of NaHCO₃ (250 mL). Both phases were separated and the aqueous phase was extracted with diethyl ether (5×200 mL). The combined organic phases were dried over Na₂SO₄, filtered and evaporated in vacuo to obtain a residual red oil (59.1 g). Distillation in vacuo (70° C., 1 Torr aprox.) afforded a mixture of 2 and 2′, as a pure colorless oil (36.5 g, 23% yield in two steps) with a cis/trans proportion of 4:10 (¹H-NMR).

### Analytical Data of 2 (Trans Isomer)

¹H-NMR (200 MHz, CDCl₃) δ: 1.30 (t, J=7.2 Hz, 3H, 2″-H), 2.22 (s, 3H, 2′-H), 4.21 (q, J=7.2 Hz, 2H, 1″-H), 5.72 (d, J=12.6 Hz, 1H, 2-H), 8.30 (d, J=12.6 Hz, 1H, 3-H).

### Analytical Data of 2′ (Cis Isomer)

¹H-NMR (200 MHz, CDCl₃) δ: 1.30 (t, J=7.2 Hz, 3H, 2″-H), 2.28 (s, 3H, 2′-H), 4.20 (q, J=7.4 Hz, 2H, 1″-H), 5.30 (d, J=7.3 Hz, 1H, 2-H), 7.54 (d, J=7.3 Hz, 1H, 3-H).

### Isomerization of the cis/trans mixture (2/2′) to ethyl trans-β-acetoxyacrylate (2)

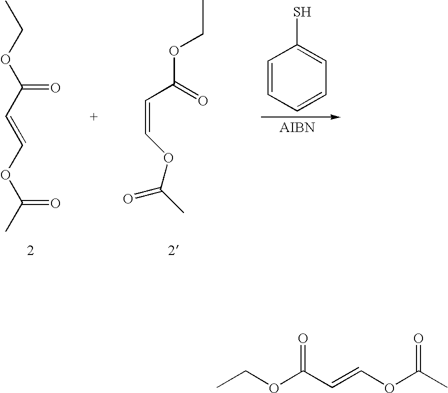

In a well-closed flask, under magnetic stirring, the 2/2′ mixture obtained from several operations (52.5 g, 39:100 cis/trans proportion, 332 mmol) was treated with thiophenol (16.3 ml, 17.5 g, 159 mmol) and 2,2′-azobis(2-methylpropionitrile) (AIBN, 8.31 g, 50.6 mmol) and the mixture was heated to 80° C. for 2.5 hours. The flask was cooled for 2 hours and the crude was diluted with ethyl acetate (400 mL) and washed with an aqueous solution of NaOH 0.01 N (400 mL). The organics were dried over Na₂SO₄, filtered and evaporated in vacuo to leave a pale yellow oil. Distillation in vacuo (53° C., 0.5-1.0 Torr) afforded 2 (55.8 g, quantitative yield) with a cis/trans proportion of 3:97 (¹H-NMR), slightly contaminated with aromatic sulphurated products.

### Preparation of (E)-1-methoxy-3-trimethylsilyloxy-1,3-butadiene (4)

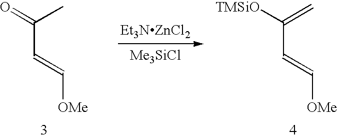

Under an inert atmosphere anhydrous ZnCl₂ (2.52 g, 18.5 mmol) was slowly added under magnetic stirring to triethylamine (distilled over KOH) (145 g, 200 mL, 1.43 mol). The mixture was stirred for 1 hour at room temperature until a fine suspension was obtained. A solution of compound 3 (63.1 g, 630 mmol) in toluene (190 mL) was then added over 5 min, followed by gradual addition of chlorotrimethylsilane (137.0 g, 160 mL, 1.26 mol) over a period of 10 min. An exothermic reaction was noted. After 30 minutes, the temperature was raised to 40° C. and stirring was continued overnight. Following cooling, the reaction mixture was diluted with diethyl ether (1 L), filtered and washed with diethyl ether (4×100 mL). The combined filtrate and ether washings were concentrated in vacuo to leave a brown oil. Distillation trough a Vigreux column (52° C., 1.0 Torr) afforded compound 4 in a middle cut, slightly contaminated with compound 3 [80.1 g, 91% purity (¹H-NMR), 67% yield of 4].

NOTE: compound 4 is commercially available (e.g. Aldrich®).

### Analytical Data of 4

¹H-NMR (200 MHz, CDCl₃) δ: 0.23 [s, 9H, OSi(CH₃)₃], 3.59 (s, 3H, OCH₃), 4.09 (d, J=8.2 Hz, 2H, 4-H), 5.35 (d; J=512.2 Hz, 1H, 2-H), 6.83 (d, J=12.2 Hz, 1H, 3-H).

### Diels-Alder Adduct of 2 and 4

### 5-O-acetyl-4-ethoxycarbonyl-3-O-methyl-1-O-trimethylsilyl-cyclohexen-1,3,5-triol

### [(±) 5a+5b]

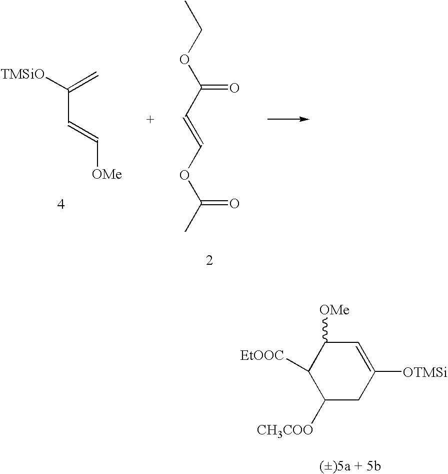

In a 250 mL round bottom flask a small amount of hydroquinone (372 mg) was added under magnetic stirring to a mixture of the Danishefky diene [4, 72.9 g, 91% purity (¹H-NMR), 385 mmol] and 2 (55.8 g, cis/trans 3:97, 353 mmol) and the mixture was heated at 180° C. for 1.5 hours. An additional amount of 372 mg of hydroquinone was added and the reaction mixture was distilled in vacuo (94° C., 3.0×10⁻² mm Hg) to afford a slightly contaminated mixture of (±) 5a+5b (72.0 g, 62% yield), with the substituents at the 4- and 5-position oriented in trans.

NOTE: Upon increasing the temperature of the distillation bath to 170° C. or higher, different quantities of the phenolic derivative 6 are obtained. The phenol derivative 6 likewise is obtained as the main isolated product when purification on silica gel is undertaken. The addition of fresh hydroquinone right before the distillation seems to avoid the formation of 6. Compound 6 could not detected by NMR when using this improved procedure.

### Representative Analytical Data for the Major Derivative (±) 5a (Substituents at 3 and 4 in Trans)

¹H NMR (CDCl₃) δ 0.21 (s, 9H), 1.27 (t, 3H, J=7.3 Hz), 2.01 (s, 3H), 2.19 (m, 1H), 2.55 (dd, 1H, J=16.7, 5.5 Hz), 2.77 (dd, 1H, J=11.4, 8.4 Hz), 3.31 (s, 3H), 4.20 (m, 2H), 4.35 (dm, 1H, J=8.4 Hz), 4.94 (t, 1H, J=2.2 Hz), 5.13 (ddd, 1H, J=11.0, 9.2, 5.9 Hz).

¹³C NMR (CDCl₃) δ 0.06 (q), 14.2 (q), 20.8 (q), 35.4 (t), 51.1 (t), 55.4 (q), 60.9 (t), 68.8 (d), 76.5 (d), 103.3 (d), 149.3 (s), 170.0 (s), 172.2 (s).

### Ethyl p-hydroxybenzoate (6) analytical data

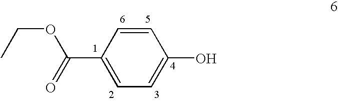

¹H-NMR (200 MHz, CDCl₃) δ: 1.39 (t, J=7.2 Hz, 3H, —CH₂CH₃), 4.37 (q, 2H, —CH₂CH₃), 6.91 [d, J=8.9 Hz, 2H, 3(5) —H], 7.36 (broad s, 1H, 4-OH), 7.96 [d, J=8.9 Hz, 2H, 2(6) —H].

¹³C-NMR (50.3 MHz, CDCl₃) δ: 14.1 (—CH₂CH₃), 61.1 (—CH₂CH₃), 115.3 [C3(5)], 122.2 (C1), 132.0 [C2(6)], 160.7 (C4), 167.6 (C═O).

### (±) 4-hydroxymethyl-cyclohex-2-en-1,5-diol (7a)

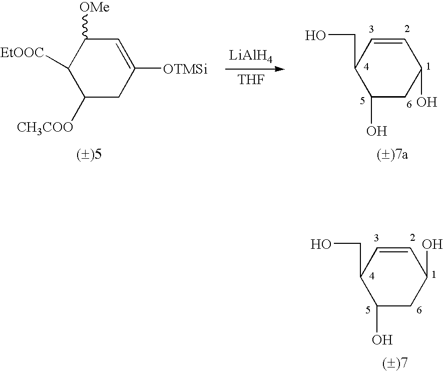

In a 1 L three necked bottom flask on a ice-NaCl bath, a suspension of LiAlH₄ (25.0 g, 658 mmol) in dry THF (220 mL) was prepared under magnetic stirring in an inert atmosphere. To this cooled suspension, a solution of the impure mixture of 5a+5b (27.2 g) in dry THF (85 mL) was added dropwise during 30 minutes. After stirring at 0° C. for 2 hours, the reaction was continued at room temperature for an additional 19 hours. The mixture became very viscous and was diluted with dry THF (110 mL). After cooling on an ice-NaCl bath, the mixture was treated consecutively and very carefully (equipping the system with a good gas-exit) with water (25 mL), stirring for 15 minutes, with 15% aqueous NaOH (25 ml), stirring for 15 minutes more, and finally with more water (75 ml). A dry granular precipitate was produced, which was easy to filter and wash. The suspension was stirred for 30 minutes and the precipitate was filtered over a layer of Celite®, and washed with water (5×100 mL) and ethyl acetate (3×100 mL). Both phases were separated and the aqueous phase was washed with ethyl acetate (3×100 mL). The aqueous phase was evaporated to dryness to give a brown gummy residue (21.1 g) which was filtered through a silica gel column (210 g) packed with ethyl acetate, eluting with mixtures of EtOAc/MeOH of increasing polarity. The title product 7a was isolated as a pale yellow oil (3.55 g, 24.7 mmol, 30%), preceded by its epimer 7b (6.44 g) as an impure mixture.

### Analytical Data of 7a

¹H NMR (CDCl₃+DMSO-d₆) δ 1.48 (td, 1H, J=11.3, 9.2 Hz), 2.02-2.23 (m, 2H), 3.35 (m, 1H), 3.61 (m, 2H), 3.75 (d, 1H, J=5.8 Hz, OH), 4.01 (t, 1H, J=4.6 Hz, OH), 4.11 (m, 1H), 4.20 (d, 1H, J=3.3 Hz, OH), 5.25 (dt, 1H, J=9.9, 2.0 Hz), 5.58 (dm, 1H, J=9.9 Hz).

¹³C NMR (CDCl₃+DMSO-d₆) δ 39.7 (t), 45.8 (d), 65.2 (t), 65.9 (d), 69.4 (d), 126.1 (d), 132.4 (d).

LISMS (THGLY/NaOAc) 167 (M+Na)⁺ (C₇H₁₂O₃)

Data of 7b:

¹H NMR (DMSO-d₆) δ 1.37 (td, 1H, J=11.7, 9.9 Hz), 1.92-2.10 (m, 2H), 3.24-3.45 (m, 2H), 3.63 (dt, 1H, J=10.2, 4.4 Hz), 4.07 (m, 1H), 4.49 (t, 1H, J=5.3 Hz, OH), 4.63 (d, 1H, J=5.1 Hz, OH), 4.70 (d, 1H, J=5.9 Hz, OH), 5.52 (d, 1H, J=11.0 Hz), 5.57 (d, 1H, J=11.0 Hz).

¹³C NMR (DMSO-d₆) δ 42.0 (t), 47.2 (d), 62.2 (t), 65.9 (d), 66.3 (d), 127.7 (d), 132.8 (d).

LISMS (THGLY/TFA) 145 (M+H)⁺ (C₇H₁₂O₃)

### Formation of the Ketal of 7a

### (±) 5,7-O-benzylidene-4-hydroxymethyl-cyclohex-2-en-1,5-diol (8)

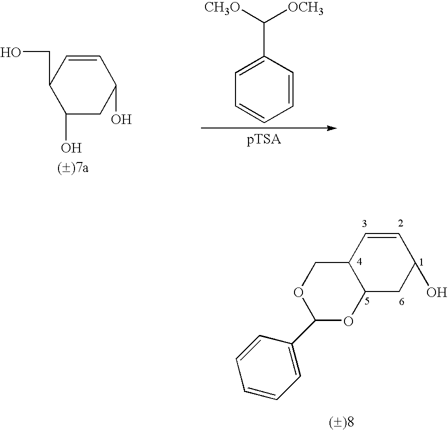

Under an inert atmosphere benzaldehyde dimethyl acetal (6.2 mL, 41.2 mmol) and p-toluenesulfonic acid monohydrate (300 mg, 1.58 mmol) were added to a solution of (±)₇a (4.49 g, 31.1 mmol) in dry dioxane (140 mL). The mixture was stirred at room temperature for 24 hours and subsequently poured into ethyl acetate (100 mL), washed with water (250 mL), dried over Na₂SO₄ and concentrated to give a white residue (8.91 g). Chromatographic purification on silica gel (270 g) eluting with mixtures of hexane/EtOAc of increasing polarity afforded the desired product 8 as a white crystalline solid (5.06 g, 70% yield, 80% yield based on recovered 7a).

The aqueous phase was evaporated to dryness, to recover the starting material 7a (600 mg, 13% recovery).

### Analytical Data of 8a

¹H-NMR (500 MHz, CDCl₃) δ: 1.60 (d, J=7.3 Hz, 1H, OH), 1.80 (ddd, 1H, 6-Hₐ), 2.53 (ddd, 1H, 6-Hₑ), 2.60 (m, 1H, 4-H), 3.61 (t, J=11.2 Hz, 1H, 7-Hₐ), 3.68 (ddd, 1H, 5-H), 4.26 (dd, J=10.7 and 4.4 Hz, 1H, 7-Hₑ), 4.53 (m, 1H, H-1), 5.42 (ddd, J=9.7 Hz, 1H, 2-H), 5.59 (s, 1H, PhCH), 5.74 (ddd, J=9.8 Hz, 1H, 3-H), 7.31-7.40 and 7.48-7.53 (m, 5H, arom-H).

¹³C-NMR (500 MHz, CDCl₃) δ: 38.6 (C-6), 40.1 (C-4), 68.0 (C-1), 70.7 (C-7), 77.7 (C-5), 102.2 (PhCH), 125.0 (C-2), 126.2 (ar-C₀), 128.3 (ar-Cₘ), 129.0 (ar-Cᵢ), 132.7 (C-3), 138.1 (ar-Cₚ).

LISMS (GLY/TFA) 233 (M+H)⁺ (C₁₄H₁₆O₃)

Additional amounts of the desired 8a can be obtained using the other epimer 7b, using an oxidation-reduction cycle as outlined below.

Therefore, the crude 7b (2.3 g, 14.58 mmol) was treated with benzaldehyde dimethyl acetal (3.28 mL, 21.87 mmol) in the presence of p-toluenesulfonic acid monohydrate (PTSA, 138 mg, 0.73 mmol) in 1,4-dioxane (30 mL) at r.t. for two days. Ice was added and the mixture was stirred at r.t. for 0.5 hr and extracted with EtOAc (3×). The combined organic solvents were washed with water and brine, dried over sodium sulfate and concentrated. The residue was purified on silica gel (hexane/EtOAc 1:1) to afford a mixture of 8b and 8a (3:1, 1.2 g) as a light yellow solid.

The mixture of 8a/8b (3:1, 415 mg, 1.79 mmol) and MnO₂ (1.56 g, 17.9 mmol, 10 eq) in dry CH₂Cl₂ (15 mL) was stirred at rt for 21 hrs. The reaction mixture was diluted with CH₂Cl₂ and filtered through Celite. The filtrate was concentrated and the residue was chromatographed on silica gel (hexane-EtOAc 2:1) to afford 9 (340 mg, 83%) as a colourless oil.

¹H NMR (CDCl₃) δ 2.65 (dd, 1H, J=16.4, 13.1 Hz), 2.83 (m, 1H), 2.95 (dd, 1H, J=16.4, 4.8 Hz), 3.79 (t, 1H, J=11.1 Hz), 4.04 (ddd, 1H, J=13.1, 9.2, 4.8 Hz), 4.45 (dd, 1H, J=11.1, 4.8 Hz), 5.63 (s, 1H), 6.13 (dd, 1H, J==9.9, 2.9 Hz), 6.58 (dd, 1H, J=9.9, 1.8 Hz), 7.39 (m, 3H), 7.51 (m, 2H).

¹³C NMR (CDCl₃) δ 39.9 (d), 44.3 (t), 69.2 (t), 77.4 (d), 101.7 (d), 126.1 (d), 128.4 (d), 129.2 (d), 132.1 (d), 137.5 (s), 144.9 (d), 196.8 (s).

LISMS (NBA) 231 (M+H)⁺. (C₁₄H₁₄O₃)

### Conversion of 9 to 8a

To a solution of 9 (340 mg, 1.5 mmol) in MeOH (15 mL) at rt was added CeCl₃.7H₂O (838 mg, 2.25 mmol, 1.5 eq). After stirring at rt for 1 hr, NaBH₄ (68 mg, 1.8 mmol, 1.2 eq) was added in portions. The reaction was stirred at rt for 2 hrs and quenched with crushed ice. The resulting mixture was stirred at rt for 0.5 hr and concentrated. The residue was taken into ethyl acetate and washed with water and brine, dried over sodium phosphate and concentrated. The residue was chromatographed on silica gel (hexane-EtOAc 5:1 and 1:1) to give 8a as a white solid which proved identical to the previous material.

The product 8a, or its analogues, either under their racemic form, or under the form of their separated isomers, as represented by the general structure III, can be used for synthesis of cyclohexenyl nucleoside analogues of general structure IV, according to standard procedures for alkylation of heterocyclic bases. Hereto, in the general structure III, R¹ and R² are representing protecting groups (e.g. R₁, R₂═C₆H₅—CH═), and R³ represents a leaving functionality (e.g. R³═SO₂CH₃, SO₂CF₃, SO₂C₆H₄CH₃, SO₂C₆H₄CH₃, SO₂C₆H₄Br) enabling nucleophilic substitution reactions, or R³ represents hydrogen, to be used in Mitsunobu reactions.

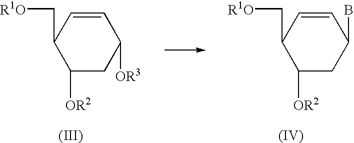

### Example:

### N²-Benzoyl-9-(5-hydroxy-4-hydroxymethyl-2-cyclohexenyl)guanine ((±) 11)

To a mixture of (±) 8a (696 mg, 3 mmol), 2-amino-6-chloropurine (1.02 g, 6 mmol) and triphenyl phosphine (PPh₃, 1.57 g, 6 mmol) in dry 1,4-dioxane (30 mL) was added slowly a solution of DEAD (945 mL, 6 mmol) in dry 1,4-dioxane (10 mL). The reaction was stirred at r.t. overnight and concentrated. The residue was taken on silica gel and chromatographed on silica gel (CH₂Cl₂/MeOH 100:1 and 50:1) to afford the crude 10 (2 g) and the N₇-epimer (140 mg) as a white solid.

The crude 10 (2 g) was treated with TFA/H₂O (3:1, 20 mL) at r.t. for 2 days. The reaction mixture was concentrated and coevaporated with toluene. The residue was chromatographed on silica gel (CH₂Cl₂/MeOH 50:1 and 10:1) to produce (±) 11 (220 mg, 27% overall yield starting from 8a).

The spectrum of 11 is identical to that previously reported.

TABLE I
Antiviral activity of D-cyclohexenyl G and L-cyclohexenyl G in comparison with approved
antiviral drugs: 50% Inhibitory concentration (IC₅₀) values are given in μg/ml.

| | D-cyclohexenyl G | | L-cyclohexenyl G | | | | | |
|---|---|---|---|---|---|---|---|---|
| Virus | Activity | Selectivity index | Activity | Selectivity index | Brivudin | Acyclovir | Ganciclovir | Cidolovir |
| HSV-1 (KOS)^{a} | 0.002^{b} | >2.10⁵ | 0.003^{b} | >5.10³ | 0.001^{b} | 0.01^{b} | 0.001^{b} | ND |
| HSV-1 (F)^{a} | 0.002^{b} | >2.10⁵ | 0.003^{b} | >5.10³ | 0.001^{b} | 0.003^{b} | 0.001^{b} | ND |
| HSV-1 (McIntyre)^{a} | 0.004^{b} | >1.10⁵ | 0.004^{b} | >4.10³ | 0.001^{b} | 0.005^{b} | 0.001^{b} | ND |
| HSV-2 (G)^{a} | 0.05^{b} | >8.10³ | 0.07^{b} | >2.2 10² | >80^{b} | 0.02^{b} | 0.002^{b} | ND |
| HSV-2 (196)^{a} | 0.07^{b} | >5.10³ | 0.1^{b} | >1.6 10² | >80^{b} | 0.02^{b} | 0.001^{b} | ND |
| HSV-2 (Lyons)^{a} | 0.07^{b} | >5.10³ | 0.07^{b} | >2.2 10² | >80^{b} | 0.02^{b} | 0.001^{b} | ND |
| HSV-1 (TK⁻KOS ACV^{f})^{a} | 0.38^{b} | >1.10³ | 1.28^{b} | >12 | >80^{b} | 9.6^{b} | 0.48^{b} | ND |
| HSV-1 (TK⁻/TK^{f} VMW1837)^{a} | 0.01^{b} | >4.10⁴ | 0.01^{b} | >1.6 10³ | >80^{b} | 0.07^{b} | 0.01^{b} | ND |
| VZV (YS)^{c} | 0.49^{d} | >40 | 1.2^{d} | >16 | 0.03^{d} | 1.1^{d} | ND | ND |
| VZV (OKA)^{c} | 0.64^{d} | >30 | 1.9^{d} | >10 | 0.003^{d} | 0.8^{d} | ND | ND |
| VZV (TK⁻07/1)^{c} | 2.1^{d} | >10 | 5.8^{d} | >3 | >20^{d} | 13^{d} | ND | ND |
| VZV (TK⁻YS/R)^{c} | 2.8^{d} | >7 | 6.8^{d} | >3 | >50^{d} | 28^{d} | ND | ND |
| CMV (AD 169)^{c} | 0.6^{d} | >30 | 1.5^{d} | >13 | ND | ND | 0.6^{d} | 0.08^{d} |
| CMV (Davis)^{c} | 0.8^{d} | >25 | 1.7^{d} | >12 | ND | ND | 0.8^{d} | 0.2^{d} |

^{a}Activity determined in E₀SM cell cultures
^{b}Minimum inhibitory concentration (μg/ml) required to reduce virus-induced cylopathogenicity by 50%
^{c}Activity determined in HEL cells
^{d}Inhibitory concentration (μg/ml) required to reduce virus plaque formation by 50%. Virus input was 20 plague forming units (PFU)
ND: not determined

TABLE II
Cytotoxicity of D-cyclohexenyl G and L-cyclohexenyl G in
four different cell lines (concentrations in μg/ml)

| Cell line | D-cyclohexenyl G | L-cyclohexenyl G | Brivudin | Acyclovir | Ganciclovir | Cidolovir |
|---|---|---|---|---|---|---|
| HeLa^{a} | 400 | 400 | ≧400 | ND | ND | ND |
| Vero^{a} | 400 | 400 | ≧400 | ND | ND | ND |
| E₈SM^{a} | >400 | >18 | ≧400 | ≧400 | >100 | ND |
| HEL^{b} | >20 | >20 | >50 | >50 | >50 | >50 |
| HEL^{c} | 11 | >20 | >200 | >200 | >50 | >50 |

^{a}Minimum cylotoxic concentration causing a microscopically detectable alteration of cell morphology
^{b}Cytotoxic concentration required to reduce cell growth by 50%

The invention claimed is:

1. A six membered, at least partially unsaturated, carbocyclic nucleoside compound, including the (−) enantiomer, the (+) enantiomer, and pharmaceutically acceptable salts and esters thereof, the compounds represented by formula I: 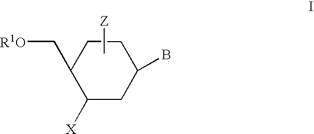
wherein:
Z represents the presence of one double bond in the six membered carbocylic ring,
B is a heterocyclic ring selected from the group consisting of pyrimidine and purine bases,
X is an azido, F, or OR²,
R¹ and R² are the same or different and represent the same or different protecting groups which are combined to form a protecting group or are each a protective group, hydrogen, alkyl, alkenyl, acyl or phosphate moieties wherein;
the alkyl moiety is a saturated, substituted or unsubstituted straight or branched chain hydrocarbon radical having from 1 to 20 carbon atoms,
the alkenyl moiety is an unsaturated congener of the alkyl group and,
the acyl moiety is an alkanoyl or aroyl moiety, wherein alkanoyl is an alkyl carbonyl radical, wherein alkyl is as described above and aroyl represents benzoyl substituted benzoyl or naphthoyl.

2. A six membered, at least partially unsaturated, carbocyclic nucleoside compound, according to claim 1, being a cyclohexenyl nucleoside compound having a formula selected from the group consisting of II and III:

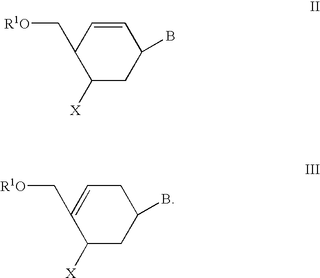

3. Compound according to claim 1, selected from the group of compounds consisting of IV, V, VI, VII, VIII, IX, X and X′:

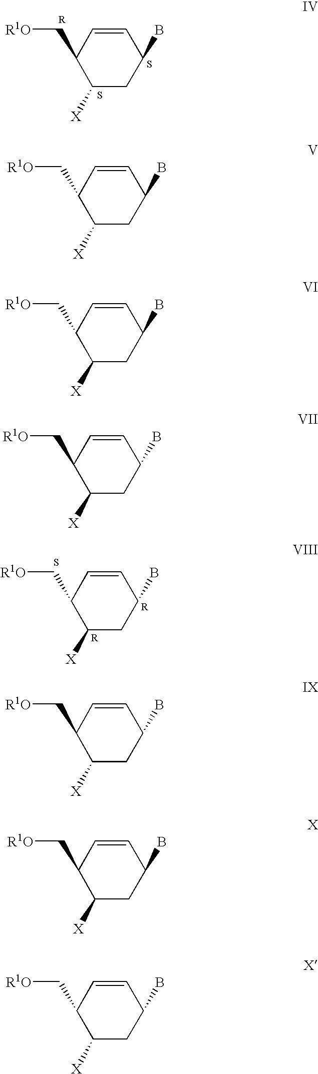

4. Compound according to claim 1, wherein the C₁ bearing B substituent and the C₅ bearing X substituent both have the (S)-configuration, and the C₄ bearing —OR¹ substituent has the (R)-configuration, as depicted by formula IV in claim 3.

5. Compound according to claim 1, wherein the C₁ bearing B substituent and the C₅ bearing X substituent both have the (R)-configuration, and the C₄ bearing —OR¹ substituent has the (S)-configuration, as depicted by formula VIII in claim 3.

6. Compound according to claim 1, wherein X is represented by a hydroxyl group in the (S)-configuration.

7. Compound according to claim 1, wherein X is hydroxyl in the (R)-configuration.

8. Compound according to claim 1, wherein B is derived from the group consisting of pyrimidine bases.

9. Compound according to claim 7, wherein the pyrimidine base has formula XI: 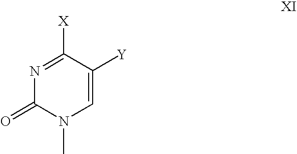
wherein X is chosen from the group consisting of:
OH, NH₂, and NHQ,
wherein;
Q is selected from the group consisting of:
OH and C₁₋₅ alkyl, and
Y is selected from the group consisting of:
H, F, Cl, Br, I, C₁₋₅ alkyl, haloethyl and CH═CH—R,
wherein R represents hydrogen, halogen or C₁₋₅ alkyl, and wherein
haloethyl contains from 1 to 4 F, Cl or Br atoms.

10. Compound according to claim 1, wherein B is selected from the group consisting of purine bases which are optionally substituted with aza, deaza, deoxy or deamino analogues, guanine, 2,6-diaminopurine, hypoxanthine and xanthine.

11. Compound according to claim 1, wherein the protecting group is selected from the group consisting of a silyl protecting group, a benzyl protecting group, a benzoyl protecting group and a C₆H₅—CH═ group.

12. Compound according to claim 1 selected from the group consisting of:
9-[(1S,4R,5S)-5-(tert-Butyldimethylsilyloxy)-4-(tert-butyldimethylsilyloxymethyl)-2-cyclohexenyl]adenine
9-[(1S,4R,5S)-5-Hydroxy-4-hydroxymethyl-2-cyclohexenyl]adenine
9-[(1S,4R,5S)-5-(tert-butyldimethylsilyloxy)-4-(tert-butyldimethylsilyloxymethyl)-2-cyclohexenyl]-2-amino-6-chloropurine
9-[(1S,4R,5S)-5-hydroxy-4-hydroxymethyl-2-cyclohexenyl]guanine
9-[(1R,4S,5R)-5-Benzoyloxy-4-benzoyloxymethyl-2-cyclohexenyl]adenine
9-[(1R,4S,5R)-5-hydroxy-4-hydroxymethyl-2-cyclohexenyl]adenine
9-[(1R,4S,5R)-5-Benzoyloxy-4-benzoyloxymethyl-2-cyclohexenyl]guanine, and
9-[(1R,4S,5R)-5-Hydroxy-4-hydroxymethyl-2-cyclohexenyl]guanine.

13. A method of producing the compound of claim 1, including, the (−) enantiomer, the (+) enantiomer, and pharmaceutically acceptable salts and esters thereof, comprising the steps of:
i) providing a cyclohexenyl compound of formula XII: 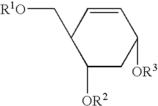
ii) substituting the OR³-group with a purine base,
wherein R¹ and R² are combined to form a protecting group or are each protecting groups and R³ is a leaving group or a Hydrogen atom, and wherein the OR³-group of the cyclohexenyl compound is substituted by a pyrimidine or purine base.

14. Process according to claim 13 wherein R³ is hydrogen and wherein a Mitsunobo reaction is utilised.

15. Process according to claim 13 wherein R³ is a leaving group enabling nucleophilic substitution.

16. Process according to claim 13, wherein the compound of formula XII has the chemical formula XIII; 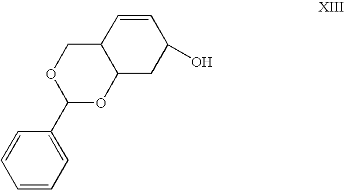
including analogues thereof either in a racemate form or separated isomers thereof.

17. Process according to claim 16, wherein compound XIII is provided by reacting (±) 4-hydroxymethyl-cyclohex-2-en-1,5 Diol of formula XIV; 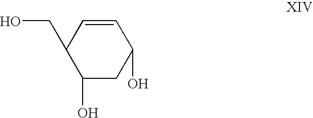
with a benzaldehyde analogue and a Lewis acid.

18. Process according to claim 13, wherein compound XIV is provided by the reduction of compound selected from the group consisting of XVA and XVB; 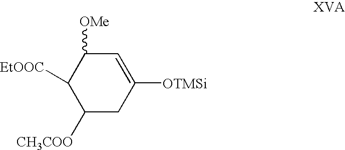 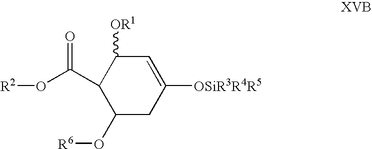
wherein for XVB:
R¹ and R² are alkyl or alkenyl moieties,
wherein:
R¹ and R² are the same or different, and
alkyl is a saturated, substituted or unsubstituted hydrocarbon radical having from 1 to 20, carbon atoms and being straight or branched chain, and
alkenyl is the unsaturated congener of the alkyl group, and
R³, R⁴ and R⁵ are alkyl, alkenyl or aryl moieties, wherein:
R³, R⁴ and R⁵ are the same or different, and
alkyl is a saturated, substituted or unsubstituted straight or branched chain hydrocarbon radical having from 1 to 20 carbon atoms and
alkenyl is the unsaturated congener of the alkyl group, and
aryl represents phenyl or substituted phenyl, and
R⁶ is an alkyl, alkenyl or acyl moiety, wherein
alkyl is a saturated, substituted or unsubstituted hydrocarbon straight or branched chain radical having from 1 to 20 carbon atoms,
alkenyl is the unsaturated congener of the alkyl group, and
acyl is an alkanoyl or aroyl moiety, wherein alkanoyl is an alkyl carbonyl radical, wherein alkyl is as described above and aroyl represents benzoyl, substituted benzoyl or naphthoyl.

19. Process according to claim 18, wherein compound XVA or XVB is provided by a Diels-Alder reaction, by the cyclo addition of a suitable diene and dienophile.

20. Process according to claim 19 wherein the diene has the following chemical structure XVI, and the dienophile has the following chemical structure XVII, wherein R¹, R², R³, R⁴, R⁵ and R⁶ are as defined in claim 20;

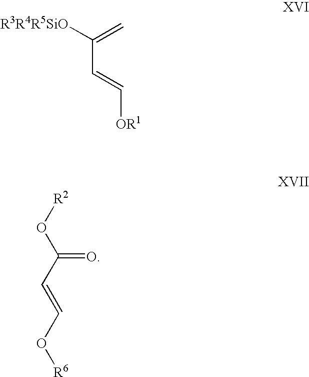

21. Process according to claim 20 wherein the diene has the chemical structure XVI′ and the dieneophile has the chemical structure XVIII;

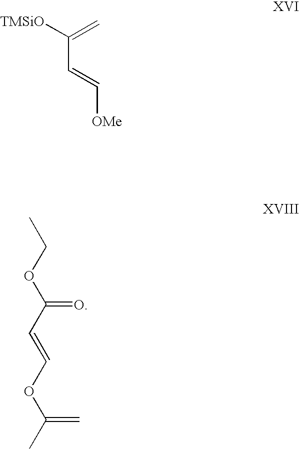

22. A six membered, at least partially unsaturated, carbocyclic nucleoside compound, including the (−) enantiomer, the (+) enantiomer, and pharmaceutically acceptable salts and esters thereof, the compounds represented by a formula selected from the group consisting of XII and XIX; 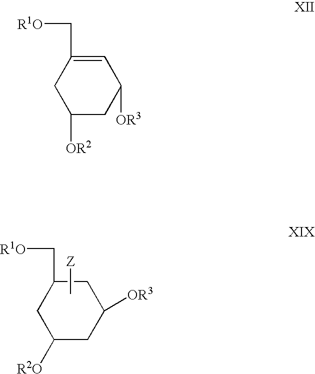
wherein:
Z represents the presence of one double bond in the carbocyclic ring,
R¹ and R² are protecting groups and R³ is a leaving group or a Hydrogen atom.

23. A cyclohexenyl compound, including the (−) enantiomer, the (+) enantiomer, and pharmaceutically acceptable salts and esters thereof, the compound represented by formula XVB; 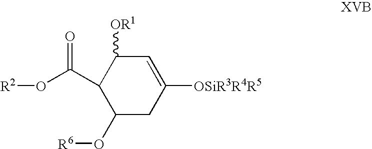
wherein R¹ and R² are alkyl or alkenyl moieties, wherein
R¹ and R² are the same or different, and
alkyl is a saturated, substituted or unsubstituted straight or branched chain hydrocarbon radical having from 1 to 20 carbon atoms,
alkenyl is the unsaturated congener of the alkyl group, and
R³, R⁴ and R⁵ are alkyl, alkenyl or aryl moieties, wherein:
R³, R⁴ and R⁵ are the same or different, and
alkyl is a saturated, substituted or unsubstituted straight or branched chain hydrocarbon radical having from 1 to 20 carbon atoms and,
alkenyl is the unsaturated congener of the alkyl group, and
aryl represents phenyl or substituted phenyl, and
R⁶ is an alkyl, alkenyl or acyl moiety, wherein:
alkyl is a saturated, substituted or unsubstituted straight or branched chain hydrocarbon radical having from 1 to 20 carbon atoms, and
alkenyl is the unsaturated congener of the alkyl group, and
acyl is an alkanoyl or aroyl moiety, wherein alkanoyl is an alkyl carbonyl radical, wherein alkyl is as described above and aroyl represents benzoyl, substituted benzoyl or naphthoyl.

24. Compound according to claim 22 selected from the group consisting of:
(4S,5R)-5-Benzoyloxy-4-benzoyloxymethyl-cyclohex-2-en-1-one,
(1S,4S,5R)-5-Benzoyloxy-4-benzoyloxymethyl-cyclohex-2-en-1-ol,
(4R,5S)-4-tert-Butyldimethylsilyloxymethyl-5-tert-butyldimethylsilyloxy-cyclohex-2-en-1-one, and
(1R,4R,5S)-5-(tert-Butyldimethylsilyloxy)-4-(tert-butyldimethylsilyloxymethyl)-cyclohex-2-en-1-ol.

25. Compound obtained by the process of claim 13.

26. Pharmaceutical composition comprising a compound and a carrier according to claim 1.

27. A pharmaceutical composition as claimed in claim 1, having antiviral activity towards herpetic viruses.

28. A pharmaceutical composition as claimed in claim 27 comprising said active ingredient in a concentration ranging from about 0.1-100% by weight.

29. A pharmaceutical composition as claimed in claim 28, having a form which is selected from the group consisting of powders, suspensions, solutions, sprays, emulsions, unguents and creams.

30. A method of providing antiviral biological activity against herpes viruses, pox viruses and related viruses, comprising administering the compound according to claim 1.

31. A method for the preparation of a pharmaceutical composition having antiviral activity against herpes viruses, pox viruses and related viruses, comprising combining the compound according to claim 1 with other ingredients.

32. Method of claim 30, wherein the biological activity comprises pharmaceutical activity.
